(19) United States
(12) Patent Application Publication
(10) Pub. No.: US 2026/0174400 A1
(43) Pub. Date: Jun. 25, 2026

(54) APPARATUS AND METHOD FOR DETECTION OF PHYSIOLOGICAL EVENTS

(71) Applicant: Strados Labs, Inc., Philadelphia, PA (US)

(72) Inventors: Yu Kan AU, Philadelphia, PA (US); Tanziyah MUQEEM, Philadelphia, PA (US); Nicholas Shane DELMONICO, Philadelphia, PA (US)

(21) Appl. No.: 19/399,655

(22) Filed: Nov. 25, 2025

Related U.S. Application Data
(63) Continuation of application No. 17/251,239, now abandoned.
(60) Provisional application No. 62/684,871, filed on Jun. 14, 2018.

(51) Int. Cl. A61B 5/00; A61B 5/0205; A61B 5/08; A61B 5/087; A61B 5/091; A61B 5/11; A61B 5/113; A61B 7/00; A61B 7/04; G10L 25/66; G16H 40/63

(52) U.S. Cl. CPC A61B 5/7282; A61B 5/0205; A61B 5/1135; A61B 5/4842; A61B 5/7275; A61B 5/7278; A61B 7/003; G10L 25/66; G16H 40/63; A61B 5/0803; A61B 5/0816; A61B 5/0823; A61B 5/087; A61B 5/091; A61B 5/1116; A61B 5/4839; A61B 7/04

(57) ABSTRACT

A method includes receiving motion data from at least one sensor of a wearable device worn by a user. The method further includes receiving audio data from at least one sensor of the wearable device, the audio data representative of sounds emanating from the user's respiratory system. The method further includes comparing the motion data to a motion data criteria. The method further includes comparing the audio data an audio data criteria. The method further includes determining, based on the comparison of the motion data to the motion data criteria and the comparison of the audio data to the audio data criteria, whether the user has coughed.

### CROSS-REFERENCE TO RELATED APPLICATIONS

This application claims priority to U.S. Provisional Application No. 62/684,871, filed Jun. 14, 2018, the entirety of which is incorporated herein by reference in its entirety.

### FIELD

The apparatuses and methods disclosed herein relate to detection of physiological events. In particular, a method and apparatus are described for acquiring sound and motion data to detect physiological events.

### BACKGROUND

Coughs serve to clear secretions from the airways and are an essential, life-sustaining physiological protective reflex. In patients with brain injuries such as stroke, spinal cord injuries, and neuromuscular diseases, impaired coughs significantly increase the risk of life-threatening pneumonia. Similarly, critically ill patients who are recently liberated from mechanical ventilation are at risk of diminished coughs.

Coughs also convey information regarding the pathophysiology of the airways and coughs associated with different diseases may have different type of characteristics. For example, coughs associated with Pertussis have a characteristic quality. Commonly, coughs may be referred to as dry coughs or wet coughs, which when evaluated in the context of other data, may confer additional information about a patient's disease.

In addition, the respiratory system produces a variety of respiratory sounds, including breath sounds and adventitious sounds. These respiratory sounds may be generated in, for example, the lungs, trachea, and mouth. While some respiratory sounds are common and are not cause for alarm, others—such as crackles, wheezes, stridor, and rhonchi—may indicate respiratory issues. Identification and characterization of these abnormal respiratory sounds may be important in providing care for patients.

Acoustic signals generated by internal body organs, such as during coughs and abnormal respiratory sounds, are transmitted to the skin, causing skin vibration. Stethoscopes are designed to capture body sounds by detecting skin vibration. The stethoscope is currently employed by medical professionals to aid in the diagnosis of diseases by listening to body sounds and recognizing the patterns associated with specific diseases. However, such use of the stethoscope is limited by the episodic nature of data acquisition as well as the limits of human acoustic sensitivity and pattern recognition. The electronic stethoscope was developed to digitally amplify the acoustic signal and aid in pattern recognition, but data acquisition is still limited by its episodic nature. Due to the weight of the stethoscope and the lack of adequate, wearable design, the electronic stethoscope is not suitable for continuous monitoring for an active user.

The advance of computer processing led to research on computerized analysis of body sounds to identify disease states. These research studies are typically conducted in a controlled setting, where sensors are used to capture body sounds for computerized analysis.

In addition, the use of wearable motion sensors, such as accelerometers, is known for monitoring various movements, such as steps.

### SUMMARY

In one aspect, a method includes receiving motion data from at least one sensor of a wearable device worn by a user. The method further includes receiving audio data from at least one sensor of the wearable device, the audio data representative of sounds emanating from the user's respiratory system. The method further includes comparing the motion data to a motion data criteria. The method further includes comparing the audio data to an audio data criteria. The method further includes determining, based on the comparison of the motion data to the motion data criteria and the comparison of the audio data to the audio data criteria, whether the user has coughed.

In another aspect, a method includes receiving motion data from at least one sensor of a wearable device worn by a user. The method further includes receiving audio data from at least one sensor of the wearable device, the audio data representative of sound emanating from the user's respiratory system. The method further includes comparing the audio data to an audio data criteria. The method further includes identifying, based on the comparison of the audio data to the audio data criteria, an abnormal respiratory sound. The method further includes determining a rate of occurrence of the abnormal respiratory sound. The method further includes assessing, based on the motion data, the activity level of the user. The method further includes determining whether a condition suffered by the user has improved or degraded.

In another aspect, a method includes receiving motion data from at least one sensor of a wearable device worn by a user. The method further includes receiving audio data from at least one sensor of the wearable device, the audio data representative of sound emanating from the user's respiratory system. The method further includes comparing the audio data to an audio data criteria. The method further includes identifying, based on the comparison of the audio data to the audio data criteria, an abnormal respiratory sound. The method further includes determining, based on the motion data, whether the abnormal respiratory sound occurred during an inspiratory portion of a respiratory cycle or an expiratory portion of a respiratory cycle.

In another aspect, a method includes receiving motion data from at least one sensor of a wearable device worn by a user. The method further includes receiving audio data from at least one sensor of the wearable device, the audio data representative of sound emanating from the user's respiratory system. The method further includes calculating, based on the motion data, the user's chest wall motion. The method further includes determining, based on the audio data, an airflow in the user's lung. The method further includes calculating the tidal volume of the user's respiratory cycle based on the chest wall motion and the airflow.

In another aspect, a wearable device includes at least one sensor configured to generate motion data in response to movement of a user, at least one sensor configured to generate audio data in response to sounds emanating from the user's respiratory system, and a processor. The processor is operable to compare the motion data to a motion data criteria and compare the audio data an audio data criteria. The processor is further operable to determine, based on the comparison of the motion data to the motion data criteria and the comparison of the audio data to the audio data criteria, whether the user has coughed.

In another aspect, a wearable device includes at least one sensor configured to generate motion data in response to movement of a user, at least one sensor configured to generate audio data in response to sounds emanating from the user's respiratory system, and a processor. The processor is operable to compare the motion data to a motion data criteria and compare the audio data an audio data criteria. The processor is further operable to identify, based on the comparison of the audio data to the audio data criteria, an abnormal respiratory sound. The processor is further operable to determine a rate of occurrence of the abnormal respiratory sound. The processor is further configured to assess, based on the motion data, the activity level of the user. The processor is further configured to determine whether a condition suffered by the user has improved or degraded.

In another aspect, a wearable device includes at least one sensor configured to generate motion data in response to movement of a user, at least one sensor configured to generate audio data in response to sounds emanating from the user's respiratory system, and a processor. The processor is operable to compare the audio data to an audio data criteria. The processor is further operable to identify, based on the comparison of the audio data to the audio data criteria, an abnormal respiratory sound. The processor is further operable to determine, based on the motion data, whether the abnormal respiratory sound occurred during an inspiratory portion of a respiratory cycle or an expiratory portion of a respiratory cycle.

In another aspect, a wearable device includes at least one sensor configured to generate motion data in response to movement of a user, at least one sensor configured to generate audio data in response to sounds emanating from the user's respiratory system, and a processor. The processor is operable to calculate, based on the motion data, a user's chest wall motion. The processor is further operable to determine, based on the audio data, an airflow in the user's lung. The processor is further operable to calculate the tidal volume of the user's respiratory cycle based on the chest wall motion and the airflow.

### BRIEF DESCRIPTION OF THE DRAWINGS

FIG. 1A is an exploded view of a wearable device in accordance with a first exemplary embodiment.

FIG. 1B is an exploded view of the diaphragm, diaphragm seal, and bottom housing/chestpiece assembly in accordance with a first exemplary embodiment.

FIG. 2A-2H are perspective views that illustrate various components of the wearable device illustrated in FIG. 1A.

FIG. 3 is a side view of the electronic components illustrated in FIG. 2C in accordance with an exemplary embodiment.

FIG. 4 is a block diagram of a body sound acquisition circuit in accordance with an exemplary embodiment.

FIG. 5 is a block diagram of sensors in accordance with an exemplary embodiment.

FIG. 6 is a block diagram of a data processing unit in accordance with an exemplary embodiment.

FIG. 7 is a flow chart diagram that illustrates steps that may be performed in accordance with an exemplary embodiment.

FIG. 8 is a flow chart diagram that illustrates data processing to determine when an abnormal respiratory sound has been captured.

FIG. 9 shows an exemplary sample of two microphone channels overlaid.

FIG. 10 shows the data from FIG. 9 after subtracting the second signal from the first signal.

FIG. 11 shows the data from FIG. 10 in a histogram format.

FIG. 12 shows the data from FIG. 10 after squaring the data.

FIG. 13 shows an exemplary sample of the motion sensor module.

FIG. 14 shows an exemplary plot of the movement of the chest wall during a single breath.

FIG. 15 shows exemplary spectrograms acquired by a first and second microphone of the wearable device.

FIG. 16 is a flow chart diagram that illustrates steps that may be performed in accordance with an exemplary embodiment.

FIG. 17 is a view of electronic components in accordance with an exemplary embodiment.

FIGS. 18 and 19 illustrate methods of determining the aspiration risk associated with a cough.

FIG. 20 illustrates a method of determining the risk associated with a cough.

FIG. 21 illustrates a method of determining cough characteristics.

FIG. 22 illustrates another method of identifying a risk level associated with a cough.

FIG. 23 illustrates a method for assessing the risk associated with an abnormal respiratory sound.

FIG. 24 illustrates another method of characterizing abnormal respiratory sounds.

### DETAILED DESCRIPTION

This description of the exemplary embodiments is intended to be read in connection with the accompanying drawings, which are to be considered part of the entire written description. The drawing figures are not necessarily to scale and certain features may be shown exaggerated in scale or in somewhat schematic form in the interest of clarity and conciseness. In the description, relative terms such as “horizontal,” “vertical,” “up,” “down,” “top” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. The term “operatively connected” is such an attachment, coupling or connection that allows the pertinent structures to operate as intended by virtue of that relationship.

The devices and methods described herein are designed for the continuous acquisition of body sounds and motion data for computerized analysis. In contrast, existing devices for body sound acquisition are designed for episodic acquisition of body sounds for human hearing. This difference leads to design differences in construction materials, weight, and mechanisms of body sound acquisition. Specifically, existing designs typically require an operator to manually press the stethoscope against the skin for adequate acoustic signal acquisition. Such data acquisition is episodic, as it is limited by the duration an operator can manually press the stethoscope against the skin. The device described herein is pressed against the skin using a mechanism such as adhesives or a clip to a piece of clothing worn by the patient. As such, data acquisition can occur continuously and independent of operator effort.

Existing mechanisms of body sound acquisitions include contact microphones, electromagnetic diaphragms, and air-coupler chestpieces made of metal.

Using electronic contact microphones and electromagnetic diaphragms for body sound acquisition is desirably accomplished via tight contact between the device and the skin. Minimal movements between the device and the skin can distort the signal significantly. Thus, the use of adhesive and a clip as attachment mechanisms may be precluded in these cases, as these attachment mechanisms do not offer sufficient skin contact for these types of body sound acquisition mechanisms.

Further, the use of electromagnetic diaphragms requires more battery power in the case of continuous monitoring, which renders the design less desirable in wearable devices.

Body sound acquisition using an air-coupler chestpiece is more forgiving with looser skin-device contact and unwanted movements. High density materials, such as metal, are used in its construction for better sound quality for human hearing. However, metallic chestpieces are too heavy for wearable applications. For example, the Littmann 3200 Electronic Stethoscope chestpiece weighs 98 grams, while an exemplary embodiment of the wearable device described herein weighs 25 grams because lightweight, lower density polymeric materials, such as acrylonitrile butadiene styrene (ABS), are used. Metals that are commonly used in chestpieces include aluminum alloy in low-cost stethoscopes and steel in premium stethoscopes. Aluminum alloys have a density of approximately 2.7 gram/cm\hat{ }3, while steels have a density of approximately 7.8 gram/cm\hat{ }3. In contrast, ABS has a density of approximately 1 gram/cm\hat{ }3. The use of a lightweight, lower density air-coupler chestpiece renders sound quality relatively poor for human hearing, but more than sufficient for computerized analysis.

Although this description of devices and methods refer to embodiments in which specific components and techniques are used to, for example, gather audio and motion data, it should be understood that other components and techniques may be used. For example, in one embodiment, a device includes a contact accelerometer that is used to capture both audio and motion data. For example, the device may include a contact accelerometer configured as described by Gupta et al. in Precision High-Bandwidth Out-of-Plane Accelerometer as Contact Microphone for Body-Worn Auscultation Devices, Solid-State Sensors, Actuators and Microsystems Workshop, Jun. 3-7, 2018, which is incorporated herein by reference in its entirety, and used in accordance with the techniques described therein.

Additionally, some embodiments of the wearable devices described herein incorporate motion sensors that acquire additional physiological data used to optimize computerized body sound analysis. The physiological data include but are not limited to the phases of respiration, i.e., inhalation and exhalation, heart rate, and the degree of chest wall expansion, as described herein.

The methods and apparatuses described herein enable respiration of a patient to be evaluated. In accordance with an exemplary embodiment, evaluation of the patient may lead, for example, to detection of medical issues associated with respiration of a patient. The evaluation may also lead to detection of worsening lung function in patients. Exemplary patients include asthmatics and patients with chronic obstructive pulmonary disease (COPD). In another exemplary application, lung sound monitoring is used to detect accumulation of fluid in the lungs due to heart failure. In another exemplary application, continuously monitoring of lung sound during diuretic therapy for heart failure is used to monitor treatment effect.

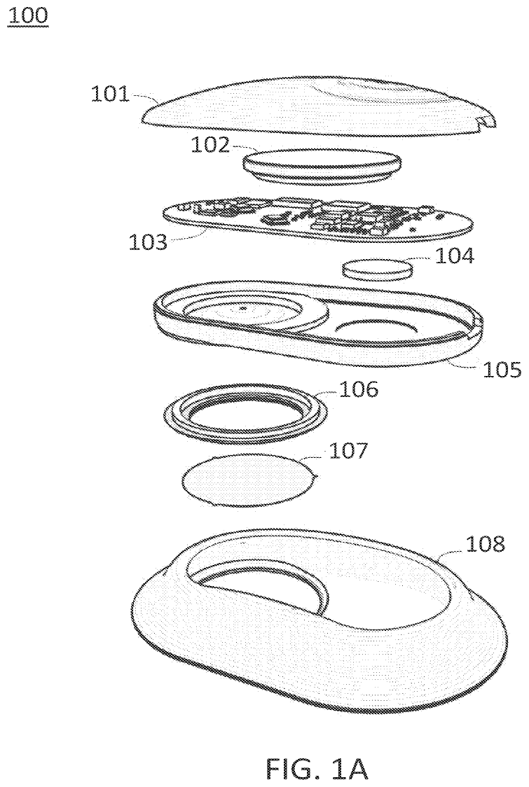

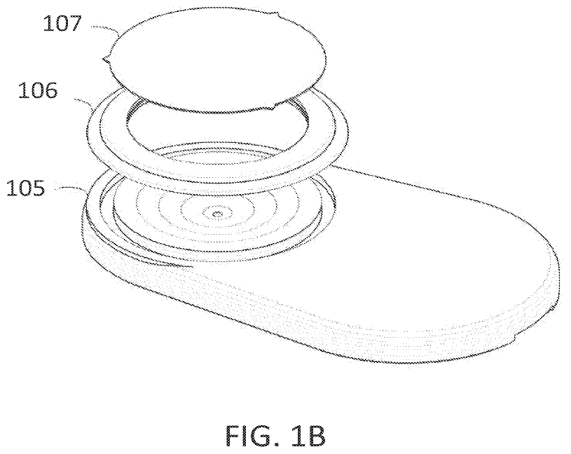

According to one embodiment, a wearable device is placed in contact with a patient's body in order to receive and process sound emanating from inside the patient's body and collect motion data. An exploded view of an exemplary wearable device 100 is illustrated in FIG. 1A. Diaphragm 107 is configured to be placed in contact with a patient's skin. A diaphragm seal 106 secures the diaphragm 107 in place. Chestpiece and bottom housing 105 is placed above diaphragm 107. Electronic components 103 are placed above chestpiece 105. Top housing 101 is placed above the electronic components 103. Soft Enclosure 108 is placed below chestpiece and bottom housing 105. Several of these components are also shown in FIG. 1B. Each component of wearable device 100 will be discussed in turn.

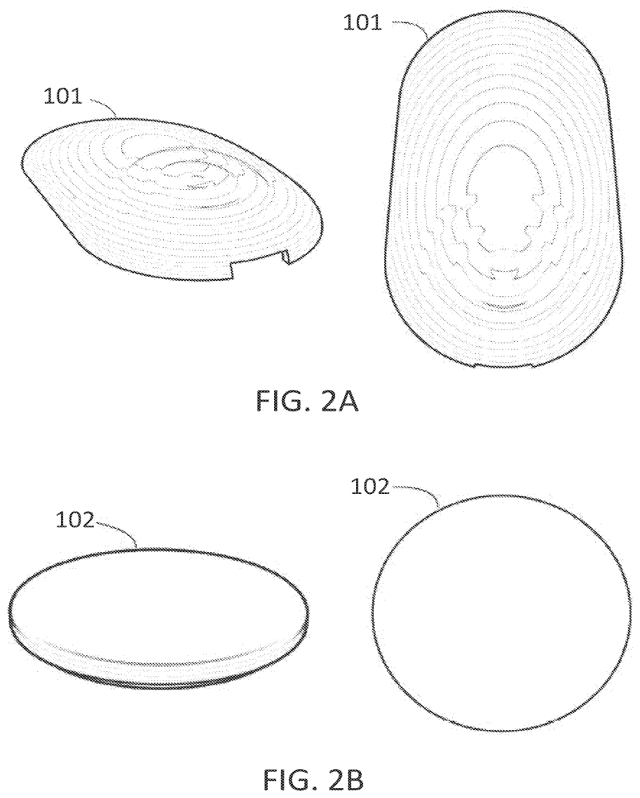

FIG. 2A illustrates exemplary top housing 101. Top housing 101 is desirably comprised of rigid, lightweight polymeric material, although other materials may be used. An exemplary size for top housing 101 is 56 mm in length, 34 mm in width, and 7 mm in height.

FIG. 2B illustrates exemplary battery 102. Battery 102 includes exemplary dimensions of 24.5 mm in diameter and 3.3 mm in height.

FIG. 2C illustrates exemplary electronic components 103 that includes exemplary dimensions of 51 mm in length, 28 mm in width, and 2 mm in height. Electronic components 130 receive audible sounds from a patient and generate data that may be used to diagnose respiratory issues. Exemplary structures and methods of operation of electronic components 103 are described in detail below.

FIG. 2D illustrates exemplary charge coil 104 that includes exemplary dimensions of 11 mm in diameter and 1.4 mm in height. Charge coil 104 enables wireless charging.

FIG. 2E illustrates exemplary bottom housing and chestpiece 105 that includes exemplary dimensions of 56 mm in length, 34 mm in width, and 4.5 mm in height. Bottom housing and chestpiece 105 is desirably comprised of rigid, lightweight polymeric material, although other materials may be used. Bottom housing and chestpiece 105 is desirably comprised of one type of material although it may be melded into one piece from several types of materials.

FIG. 2F illustrates exemplary diaphragm seal 106 that includes exemplary dimensions of 29 mm in diameter and 2.75 mm in height. Diaphragm seal 106 secures diaphragm 107 to the bottom housing and chestpiece 105.

FIG. 2G illustrates exemplary diaphragm 107 that includes exemplary dimensions of 24 mm in diameter and 0.25 mm in height. Diaphragm 107 is desirably comprised of rigid, lightweight polymeric material, although other materials may be used.

FIG. 2H illustrates exemplary soft enclosure 108. Soft enclosure 108 is desirably comprised of soft silicone and includes a bottom edge designed to hold it in place. Exemplary dimensions include a length of 72 mm, a width of 50 mm, and a height of 12 mm. Soft enclosure 108 may be designed to be affixed to a patient's skin using adhesive, although other mounting mechanisms (i.e. straps or clips) may also be used.

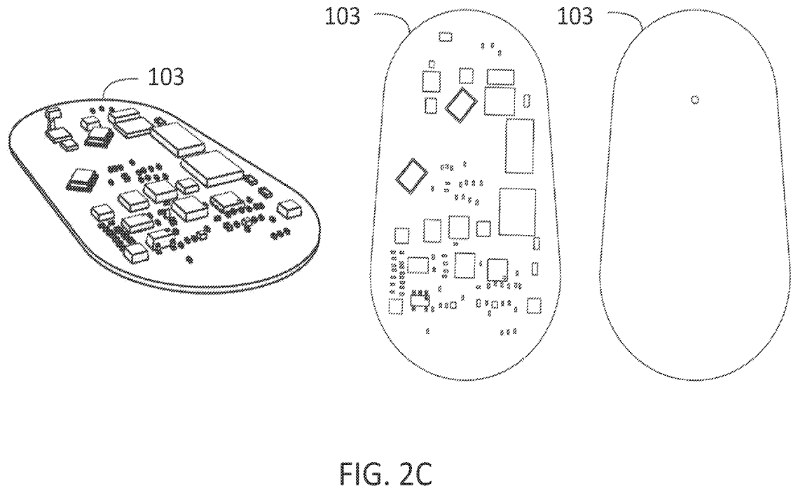

FIG. 3 provides further details regarding electronic components 103. Electronic components 103 include chest facing microphone 305. An optional background microphone 310 can be mounted on either side of electronic components 103. The microphone port hole of chest facing microphone 305 faces bottom housing and chestpiece 105. The microphone port hole of optional background microphone 310 faces top housing 101. Other parts included with electronic components 103 may be mounted on either side of electronic components 103 depending upon space availability. Battery 102 is included in order to power electronic components 103. Battery 102 may be a disc battery, for example, in order to provide electronic components 103 with a desirable outer thickness. Processor 170 is able to perform various operations as described below. Multi-sensor module 315 includes optional sensors including but not limited to motion sensors, thermometer, and pressure sensors. Power management device 320 optionally controls power levels within electronic components 103 in order to conserve power. RF amplifier 325 and antenna 330 optionally enable electronic components 103 to communicate with an external computing device wirelessly (e.g., a smartphone, tablet computer, laptop computer, cloud-based computing system, etc.). Optional USB and programming connectors 316 enable wired communication with electronic components 103.

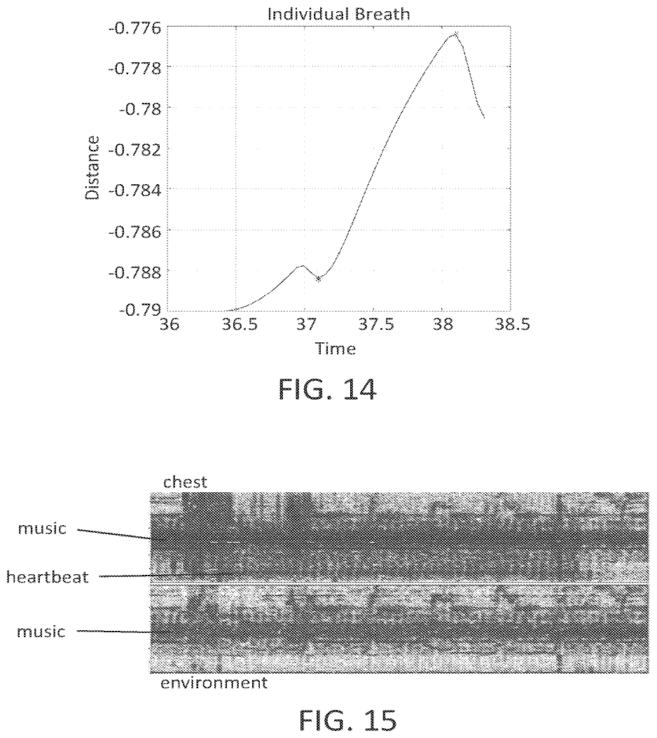

In one embodiment, multi-sensor module 315 includes a motion sensor module 317 including one or more accelerometers, a gyroscope, and a magnetometer. In one embodiment, as shown in FIG. 17, a first accelerometer and a gyroscope are provided on a first chip 321. Further, a second accelerometer and a magnetometer are provided on a second chip 322. By providing the accelerometer and the gyroscope together on the first chip 321, misalignment of the axes of the sensors is avoided. Similarly, by providing the second accelerometer and the magnetometer together on the second chip 322, misalignment of the axes of those sensors is avoided. While including multiple sensors on a single chip provides the advantages noted, in other embodiments the sensors are separately affixed to the electronics board. In one embodiment, the elements of the motion sensor module 317 can be set to collect data at a frequency of 2 kHz. In other embodiments, the elements of the motion sensor module 317 collect data at any appropriate frequency, such as 1 kHz, 2 kHz, 3 kHz, 4 kHz, or 5 kHz.

In one embodiment, the motion sensor module 317 includes four sensors. Three of the sensors are positioned such that they provide motion data in nine degrees of freedom. The fourth sensor is included to denoise the concurrent motions. In some embodiments, an accelerometer and a gyroscope (for example on first chip 321) are positioned to sense the linear and angular motion of the chest wall, this data may be used to further characterize abnormal respiratory sounds. Further, a magnetometer may be used to gather data that can be used to characterize non-chest wall motions such as walking, jumping, or ambulating with a walker, based on the linear and angular vectors of the motions. In some embodiments, an additional accelerometer may be used to gather data used to detect heart rate based on concurrent movement of the chest wall. Other applications of multi-axis motion sensing include, but are not limited to, detecting postures and specific motions during physical therapy. By placing additional motion sensors along a different axis than the motion sensors used for chest wall motion measurements, the relative contribution of each type of motion to each vector can be computed, so that multiple motions can be classified.

The data captured by motion sensor module 317 may be used to, for example, determine the amplitude of each breath, the duration of inhalation and exhalation of each breath, and the duration of the interval between breaths, as well as the variability of these parameters. Further, in users wearing more than one wearable device 100, the respiratory pattern may be further characterized by the movement of different parts of the torso, including the abdominal area and the chest wall. As will be described further herein. This information may be used in combination with the audio data captured by microphones 305, 310 to characterize abnormal respiratory sounds and assess the risks associated therewith.

The capability of detecting concurrent motions is important in peri-surgical respiratory monitoring and post-surgical physical therapy, as a patient experiencing respiratory decompensation will exhibit different types of posture and level of activity. A change in posture, chest wall movement, and ambulatory pattern (which includes but is not limited to gait, activity level, and timing of ambulation), can be used in multiple ways in a peri-surgical setting. These include but are not limited to the following: (1) detection of respiratory decompensation; (2) adjustment of medications, such as pain medications that can reduce respiratory drive; and (3) dynamic feedback for physical therapy and pulmonary rehabilitation.

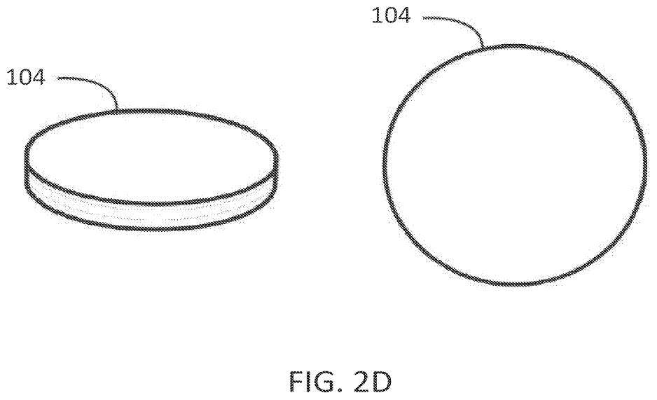

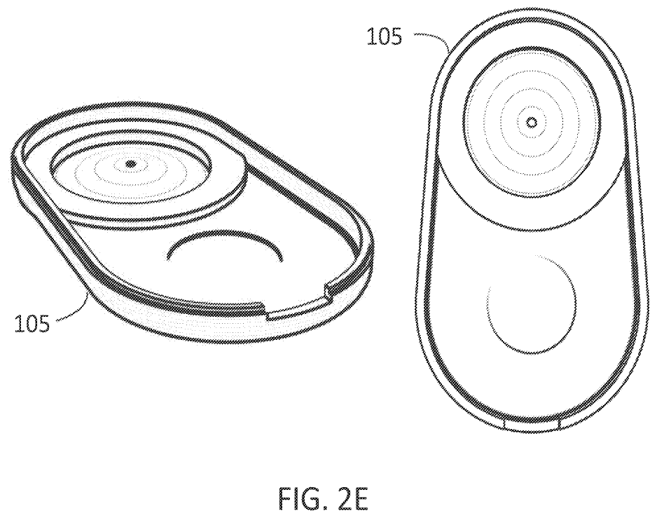

FIG. 4 is a block diagram that illustrates data acquisition circuit 150. Data acquisition circuit 150 includes sensor 160 and data processing unit 170. Received sound is received by sensor 160, which is more clearly illustrated in FIG. 5. Sensor 160 may include one or more microphones, such as, for example, a chest facing microphone 305 and optional background microphone 310 that are configured to convert acoustical energy into electrical energy. In some embodiments, one or both of chest facing microphone 305 and background microphone 310 is a capacitor-based microphone. In other embodiments, as described above, the wearable device 100 includes a contact accelerometer to capture audio data. Optional motion data, pressure data, and temperature data is also received by sensor 160, which is more clearly illustrated in FIG. 5. Sensor 160 may include multi-sensor module 315 that is configured to convert analog motion, temperature, and pressure data into electrical energy. The multi-sensor module 315 can include motion sensor module 317, a barometer 318, and a thermometer 319. Signals from each microphone 305, 310 and multi-sensor module 315 may be transmitted to A-D converter 340 and electrical bus interface 350. The data from the sensors may be processed by an on-board processor by a processor of external computer 360 (e.g., a smart phone, tablet computer, cloud-based processor, etc.).

Optional physical filter(s) 306 may also be included. Exemplary filters include linear continuous-time filters, among others. Exemplary filter types include low-pass, high-pass, among others. Exemplary technologies include electronic, digital, mechanical, among others. Optional filter(s) 306 may receive sound prior to digitization, after digitization, or both.

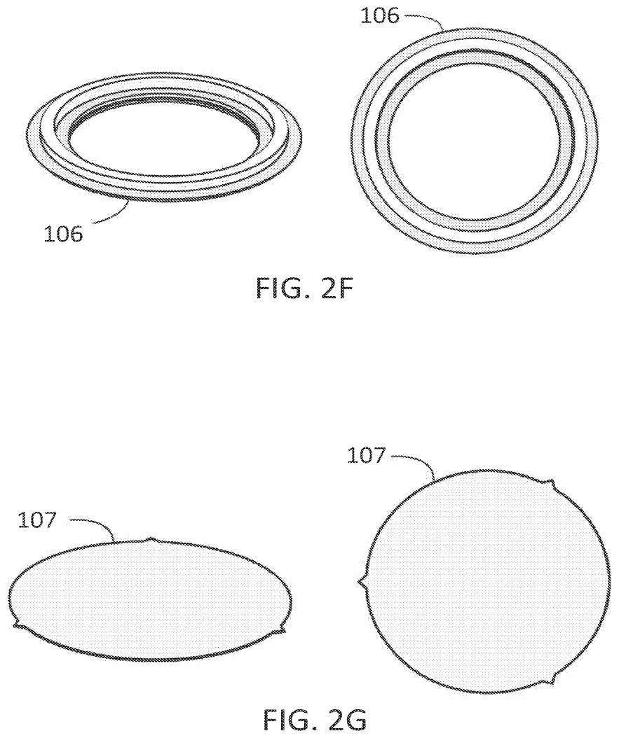

The output of electrical bus interface 350 may be transmitted to data processing unit 170, which is more clearly shown in FIG. 6. Data processing unit 170 includes digital signal processor 171, memory 172 and wireless module 173 (that includes an RF amplifier and an antenna as shown in FIG. 3). Digital signal processor 171 can be programmable after manufacturing. Exemplary processors include Cypress programmable system-on-chip, field programmable gate array with integrated features, and a wireless-enabled microcontroller coupled with a field programmable gate array. Wireless module 173 may use Bluetooth Low Energy as a wireless transmission standard. Wireless module 173 may include an integrated balun and a fully certified Bluetooth stack. Processor 171, memory 172 and wireless module 173 are desirably integrated.

In one exemplary embodiment, data is transferred from memory 172 to external computer 360. This is further described below.

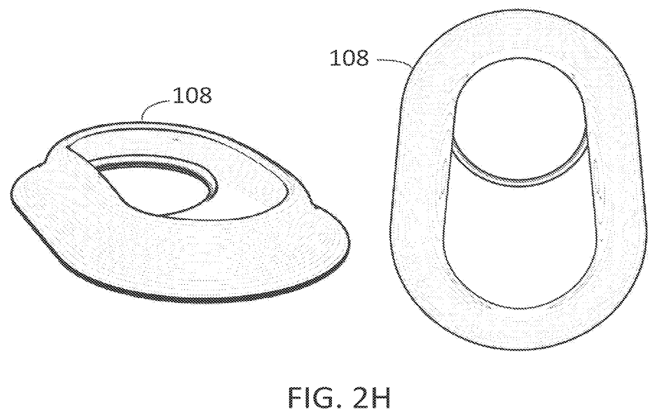

Operation of an exemplary embodiment is illustrated by FIG. 7. At step 1102, wearable device 100 is placed in contact with a patient (preferably the patient's skin). Wearable device 100 may include an adhesive to hold it in contact with the patient, although other forms of adherence may be used. Wearable device 100 is placed so that chest facing microphone 305 faces the patient and optional background microphone 310 does not face toward the patient.

At step 1104, sound from chest facing microphone 305 is acquired. At optional step 1106, sound from background microphone 310 is acquired. The sound optionally passes through filter 306 before being converted into electrical energy by microphone 305. Further, at step 1107, motion data is acquired by motion sensor module 317. After being converted to electrical energy, the audio and/or motion data passes through A-D converter 340 and electrical bus interface 350 before being received by digital signal processor 171. Processor 171 may sample the audio and/or motion data, for example at 20 kHz. Sampling may occur, for example, for twenty seconds. Step 1108 optionally includes the step of using the audio signals received at step 1106 via microphone 310 in order to perform noise cancellation. Noise cancellation may be performed using algorithms that are well known to one of ordinary skill in the art of noise cancellation.

Sampled audio and/or motion data is processed at step 1110. Audio data is processed in order to detect certain sounds associated with breathing (and/or associated with breathing difficulties). Processing at step 1110 may include, for example, Fast Fourier Transform. Processing may also include, for example, digital low pass and/or high pass Butterworth and/or Chebyshev filters. The motion data may be processed as described further herein.

At optional step 1112, data is stored in memory 172. FIG. 7 shows step 1112 performed after step 1110, but it is understood in certain circumstances that step 1112 is performed concurrently with step 1110 or prior to step 1110. There may be two types of data that are stored in memory 172. The first type of data may be the “raw” data, i.e. a recording of sounds that have been sampled by microphone 305 (and that has been subjected to noise cancellation if noise cancellation is available and desired). In one exemplary embodiment, the most recent 20 minutes of “raw” audio data is stored in memory. The data is stored in a first in, first out configuration, i.e. the oldest data is continuously deleted to make room in memory for data that is newly and continuously acquired. The second type of data that is stored in memory is processed data, i.e. data that has been subjected to a form of processing (such as time-frequency analysis) by processor 171. Examples of this type of processed data includes the examples set forth above such as Fast Fourier Transform, digital low pass and/or high pass Butterworth and/or Chebyshev filters, etc. In an exemplary embodiment, 20 seconds of processed audio data is stored in memory 172. This data is also stored in a first in, first out configuration.

At step 1114, the processed data is evaluated by processor 171 to determine if an “abnormal” respiratory sound has been captured by microphone 305. Examples of an “abnormal” respiratory sound include a wheeze, a cough, rhonchi, labored breathing, or some other type of respiratory sound that is indicative of a respiratory problem. Evaluation occurs as follows. In one exemplary embodiment, the processed data (i.e. from a transform such as a Fourier transform or a wavelet transform) results in a spectrogram. The spectrogram may correspond, for example, to the 20 seconds worth of processed data that has been stored in memory 172. The spectrogram is then evaluated using a set of “predefined mathematical features”.

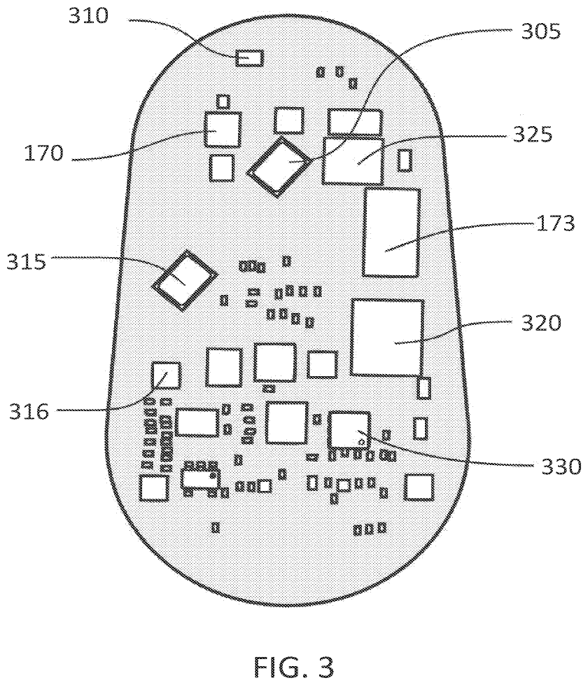

The “predefined mathematical features” are generated from multiple “predefined spectrograms”. Each “predefined spectrogram” is generated by processing data that is known to correspond to an irregular respiratory sound (such as a wheeze). A method of generating such a predefined spectrogram is illustrated by the flowchart diagram of FIG. 8 and may be performed as follows: a) a physician listens to respiratory sounds from a person using a device such as a stethoscope; b) the respiratory sounds from the person are recorded and subjected to processing such as the processing identified above; c) a spectrogram is generated based on the processing set forth above; d) the physician notes the exact time when he/she hears a sound that the physician considers to be a wheeze, e) the portion of the spectrogram that corresponds to the exact time that the physician hears the wheeze is identified, and f) that portion of the spectrogram that has been identified is used as the “predefined spectrogram.”

The predefined spectrograms can be patient specific. For example, the steps a through f above may be performed for the particular patient who will wear the wearable device 100. The predefined spectrograms can also be population based. In other words, the predefined spectrograms can be based on performing steps a through f on someone other than the patient who will wear the wearable device 100. In some embodiments, the predefined spectrograms are based on both patient specific and population based spectrograms.

Once the raw data has been acquired from the subject (step 1202), and is subject to audio processing (step 1204), spectrogram feature extraction (step 1206) may occur (shown in FIG. 8).

A set of mathematical features can be extracted from each predefined spectrogram. Mathematical feature extraction is known to one of ordinary skill in the art and is described in various publications, including 1) Bahoura, M., & Pelletier, C. (2004, September). Respiratory sounds classification using cepstral analysis and Gaussian mixture models. In Engineering in Medicine and Biology Society, 2004. IEMBS'04. 26th Annual International Conference of the IEEE (Vol. 1, pp. 9-12). IEEE; 2) Bahoura, M. (2009). Pattern recognition methods applied to respiratory sounds classification into normal and wheeze classes. Computers in biology and medicine, 39(9), 824-843; 3) Palaniappan, R., & Sundaraj, K. (2013, December). Respiratory sound classification using cepstral features and support vector machine. In Intelligent Computational Systems (RAICS), 2013 IEEE Recent Advances in (pp. 132-136). IEEE; 4) Mayorga, P., Druzgalski, C., Morelos, R. L., Gonzalez, O. H., & Vidales, J. (2010, August). Acoustics based assessment of respiratory diseases using GMM classification. In Engineering in Medicine and Biology Society (EMBC), 2010 Annual International Conference of the IEEE (pp. 6312-6316). IEEE; and 5) Chien, J. C., Wu, H. D., Chong, F. C., & Li, C. I. (2007, August). Wheeze detection using cepstral analysis in gaussian mixture models. In Engineering in Medicine and Biology Society. All of the above references are hereby incorporated by reference in their entireties.

The set of mathematical features are derived from the inherent power and/or frequency of the predefined spectrogram of data clusters using mathematical methods that include but are not limited to the following: data transforms (Fourier, wavelet, discrete cosine) and logarithmic analyses. The set of mathematical features extracted from each predefined spectrogram can vary by the method with which each feature in the set is extracted. These features may include, but are not limited to, frequency, power, pitch, tone, and shape of data waveform. See Lartillot, O., & Toiviainen, P. (2007, September). A Matlab toolbox for musical feature extraction from audio. In International Conference on Digital Audio Effects (pp. 237-244). This reference is hereby incorporated by reference in its entirety.

For example, in one embodiment, a first set of two mathematical features are extracted from a predefined spectrogram using statistical mean and mode. A second set of two mathematical features are extracted from the same predefined spectrogram using statistical mean and entropy. The set of mathematical features can also vary by the number of features in each set of mathematical features. For example, in one embodiment, a set of twenty mathematical features are extracted from a predefined spectrogram. In another example, a set of fifty mathematical features are extracted from the same predefined spectrogram. Additionally, the mathematical features may vary by the segment lengths of the predefined spectrogram with which the mathematical features are extracted. For example, a mathematical feature extracted from one-second segments of the predefined spectrogram using a statistical method is different from a mathematical feature extracted from five-second segments of the predefined spectrogram using the same statistical method.

The set of mathematical methods used to extract the “predefined mathematical features” is the “pre-specified feature extraction”. In one exemplary embodiment, the “pre-specified feature extraction” is developed using mel-frequency cepstral coefficients and is optimized using machine learning methods that include but are not limited to the following: support vector machines, decision trees, gaussian mixed models, recurrent neural network, semi-supervised auto encoder, restricted Boltzmann machines, convolutional neural networks, and hidden Markov chain (see above references). Each machine learning method may be used alone or in combination with other machine learning methods.

The “predefined mathematical features” are derived from multiple predefined spectrograms in the following manner. A feature extraction method, as defined above, is used to extract a set of mathematical features from each predefined spectrogram corresponding to a type of respiratory sound. Multiple features are evaluated in this manner. The features are then plotted together (step 1208) from multiple respiratory sound types in order to perform cluster analysis in the nth dimension (n being the number of features extracted). For example, if three features were extracted for analysis from each data file, each data file would correspond to one point in three-dimensional space, each axis representing the value of a particular feature. Thereafter, one example of algorithm generation attempts to find a hyperplane in this three-dimensional space that maximally separates clusters of points representing specific sound types. For example, if data points from wheeze files cluster in one corner of this three dimensional space while those from cough files cluster in another, a plane that separates these two clusters would correspond to an algorithm that distinguishes the two and is able to classify these sound types into two groups. This analysis can be extrapolated to as many features as needed, n, thereby moving the analysis into nth dimensional space. This allows differentiation of each sound type based on its unique feature set. The algorithm that generates outputs (sets of mathematical features) that are most similar to each other is selected as the “pre-specified algorithm” as described above. For example, ten sets of twenty statistical features is extracted from ten predefined spectrograms corresponding to wheezing using different algorithms. The algorithm that extracts ten sets of features that are the most similar to each other is selected as the “pre-specified algorithm” (step 1210). In an exemplary graphical representation of classification, lines represent the “pre-defined algorithm” in classifying data in multiple dimensions in accordance with an exemplary embodiment. Next, the “average” of the sets of mathematical features extracted with the “pre-specified algorithm” is selected as the “predefined mathematical features”. Here, “average” is defined by mathematical similarity between the “predefined mathematical features” and each set of mathematical features from which the “predefined mathematical features” derives from.

Evaluation of a spectrogram with a predefined spectrogram may be on several bases. A spectrogram is processed by the “pre-specified feature extraction” method to generate a set of mathematical features. The set of mathematical features is then compared to sets of “predefined mathematical features”, of which each set corresponds to a specific type of sound. If the similarity between the set of mathematical features extracted from a spectrogram and the predefined mathematical features of a type of respiratory sound goes past certain thresholds, then it is determined that the corresponding type of respiratory sound has been emitted. By saying ‘goes past” what may be meant is going above a value. What may alternatively be meant is going below a value. Thus, by portions of the spectrogram going above or below portions of the predefined spectrogram associated with possible abnormal respiratory sounds, it is determined that an abnormal respiratory sound may have occurred.

A variety of factors can be used to identify, from the available predefined spectrograms, those that a particular patient's data should be compared to and to otherwise classify respiratory sounds. For example, when the wearable device is used post-surgery, predefined spectrograms collected from a subject with a similar surgical anatomy can be used. Selecting appropriate comparison spectrograms in this way may provide more accurate results because general population data may be inappropriate for the post-surgery period. In some embodiments, the motion data is also compared to data gathered from patients with similar anatomy and/or suffering from similar conditions.

In addition, the appropriate predefined spectrograms can be selected based on a pulmonary disease experienced by the patient. For example, the predefined spectrograms can be filtered to those that were captured from patients with COPD. Respiratory sounds are often diminished in patients with severe COPD. COPD also affects pulmonary mechanics. The chest wall is expanded at baseline in patients with COPD, which is termed “barrel chest”. This affects angular and linear displacements, and subsequent calculation of tidal volume and airflow rate. The severity of COPD can be determined from past medical records, and for patients without adequate prior medical evaluation, from smoking history. Selecting the predefined spectrograms by matching COPD history or smoking history can help ensure that the most relevant factors are considered.

An exemplary application involves a patient with esophageal surgery, which puts the patient at high risk of chemical pneumonitis from surgical site leaks. With the development of a surgical leak, this exemplary patient's lung sound generates a specific signature. Concurrently, the patient may have increased respiratory rate and decreased tidal volume. However, the patient may have a barrel chest as a result of severe COPD. Therefore, decreased tidal volume will not result in a decrease in chest wall movement that would otherwise be expected from a patient without COPD. As described above, the predefined spectrograms may be derived from a plurality of populations, such that the difference in boundary conditions for patients with and without COPD could be gathered and applied for the exemplary case.

Additionally, the information collected by the microphones 305, 310 and/or motion sensor module 317 can be used to distinguish edematous chest wall or lungs from a chest wall and lungs that do not have an edema. This information can be used to refine or filter the spectrograms to which the patient's respiratory sounds will be compared. Because an edematous chest wall transmits sound differently than a chest wall without edema, comparison with data collected from subjects with a similar condition can further enhance the accuracy of the determination of abnormal respiratory sounds.

In addition, the predefined spectrograms can be filtered based on the patient's history of heart failure. These patients may experience wheezing due to bronchospasm or decompensated heart failure, which often also leads to an increase in weight. Based on sound alone, wheeze due to bronchospasm is hard to distinguish from a cardiac wheeze. In these patients, classification of respiratory wheezes vs. cardiac wheezes may take into account information available elsewhere in a patient's medical records. One key differentiator is a patient's past medical history. A marker of worsening heart failure is increasing body weight. This information can be used to adjust the threshold of classification. For example, in a patient without a history of heart failure, a wheeze can be classified as a wheeze due to bronchospasm regardless of the amount of weight gain. However, in a patient with heart failure, a significant weight gain (i.e., two bounds or more) will lead to the classification of a wheeze as a cardiac wheeze. Compared to patients without a history of heart failure, in patients at risk of decompensated heart failure, a smaller change in weight will lead to a classification of cardiac wheeze rather than non-cardiac wheeze.

Wheezes and other respiratory sounds can further be classified based on at what point in the respiratory cycle the wheeze occurs (e.g., during the inhalation or expiration phase). In various embodiments, it may be determined in which portion of the cycle the respiratory sound occurs based on data from motion sensor module 317 of wearable device 100, as described further herein.

In some embodiments, patient specific predefined spectrograms are acquired prior to a surgery to provide a pre-surgery benchmark for post-surgery monitoring. In addition to acquiring pre-surgery spectrograms, other pre-surgery information may be gathered. For example, the patient's chest wall movement data, heart rate, respiratory rate, and ambulatory patterns including but not limited to posture and gait. In addition to being used as benchmarks, this data can be used in the selection of appropriate boundary conditions or benchmark spectrograms for the patient. Alternatively, or additionally, the audio and/or motion data can be compared to data captured after surgery, but at an earlier time, from the same patient.

Other exemplary inputs used for selection of benchmark spectrograms or boundary conditions include video imaging inputs. The inputs could be from a camera of a personal mobile device or a “smart” television in the patient's home. Video input is used to determine the placement of the wearable device 100 on the patient's chest wall. The video may also be used to correlate sound and motion sensor data to the patient's movements, which includes but is not limited to respiration, posture, and gait. Correlation with video inputs may be incorporated into the calibration process but is not required. Video inputs from the individual may be compared against a population-based database and may contribute to selection of the appropriate boundary conditions.

Once an irregular respiratory sound (such as a wheeze) has been identified using the “predefined mathematical features” the previous 20 (for example) minutes of accumulated raw data that has been stored in memory 172 may receive “further processing.” In one exemplary embodiment, the 20 minutes of raw data is transferred from memory 172 to external computer 360 for more robust processing. In another exemplary embodiment, raw data is subjected to further processing in processor 171 without being transferred to an external computer. The further processing described above may be performed in processor 171, external computer 360, or both, depending upon respective processing power, ability to communicate wirelessly, etc.

By implementing a “further processing” step, a first algorithm is used to possibly identify an irregular respiratory sound and a second algorithm (more robust—i.e. that requires more significant processing than the first algorithm) is applied to the raw data to try to make a more accurate determination as to whether an irregular respiratory sound (such as a wheeze) has indeed occurred. In one exemplary embodiment, a first algorithm generates twenty mathematical features. A second algorithm generates fifty mathematical features and is more robust. In another exemplary embodiment, the mathematical methods used to extract each mathematical feature in the second algorithm require more processing power than the mathematical methods used in a first algorithm. As such, the second algorithm may be more robust.

Thus, this further processing may include determining whether processed data has passed (i.e. above or below) boundary conditions. The boundary conditions may include one or more of any of the inputs and/or characteristics identified above, such as the mathematical features extracted from the predefined spectrograms. In one embodiment, this is accomplished by pre-specified algorithms previously developed using a machine-learning approach using a deep-learning framework. This involves a multi-layer classification scheme. The variables used in the pre-specified algorithms in the external computer include, but are not limited to, the exemplary variables described above.

In addition to using a spectrogram with the second algorithm, other factors may also be used in the analysis. Exemplary factors include: 1) user inputs, including subjective feelings, rescue inhaler use, type and frequency of medication use, and current asthma status; 2) input from sensors (e.g., accelerometers, magnetometers, and gyroscopes) related to a patient's current physiological status, as will be described in more detail below; 3) environmental inputs available from sensors, which include but are not limited to temperature sensors and barometers; and 4) environmental inputs available from an information source such as the internet. In other words, other variables may be integrated into the analysis, in place of or in addition to the variables that form the basis of the analysis of the initial processed data (e.g., the 20 seconds of data, for example, discussed above). These factors can also include the patient's demographics, heart rate, surgical type, activity level, posture, gait, medication use, and results of medical imaging.

In one embodiment, medical imaging can be used to derive body tissue composition and anatomy. This information can then be used to define the boundary conditions to which the patient's respiratory sounds are compared.

In another embodiment, the patient's use of medication is used to further define the spectrograms and boundary conditions to which the patient's respiratory sounds are compared. Many common pain medications, including but not limited to opioids and ketamine, can cause respiratory and neurological depression. Respiratory depression may manifest with decreased tidal volume and respiratory flow rate. The wearable device 100, via the motion sensor module 317, can measure body motion and the resulting data may be used to detect these changes. Comparing the data to spectrograms of user's that are using similar medication may allow for more accurate characterization. Neurological depression may manifest with decreased tidal volume and respiratory flow rate. This condition can also manifest with aspiration and upper airway obstruction, which has an effect on lung sounds in addition to chest wall motion. Neurologic depression also leads to less overall patient movement. The wearable device 100 can measure body motion and lung sounds and the motion and audio data can be used to detect such changes. Further, in such an embodiment, the patient's medication use data can be correlated with sensor data to provide feedback on the safety of pain medication use.

The information gathered by the wearable device 100 (e.g., from the motion sensor module 317) and/or provided by a patient or caregiver (e.g., patient height, patient weight, patient demographics, medications, surgical information) can also be used to refine and adjust the boundary conditions. For example, the comparison mathematical features extracted from the predefined spectrograms may be adjusted up or down based on data derived from motion sensor module 317.

When it is determined that the data has crossed above or below the boundary conditions, an alert or warning can be provided. The alert or warning can be issued to the patient and/or to a physician or caregiver. For example, the wearable device 100 can issue audible, visual, or tactile feedback, such as by beeping, illuminating one or more lights, or vibrating. Alternatively, the wearable device 100 can be connected to a computing device, such as a smartphone, via wireless module 173. As a result, an alert can be issued on the computing device. In some embodiments, the computing device issuing the alert is the external computer 360. The alert can also be sent to a physician or other caregiver such that the caregiver can contact the patient or notify emergency responders.

The alarm threshold (i.e., the amount of deviation from the boundary conditions required to issue the alarm) may vary from patient to patient. For example, if the patient is using the wearable device 100 after surgery, the alarm threshold may be lower (i.e., more sensitive) because the patient may be at higher risk than the general population. The threshold may further vary based on the type of surgery and potential complications. For example, a patient at risk of chemical pneumonitis may require a lower threshold.

The “raw” data that may be stored, for example, in memory 172 provides multiple functions. For example, it provides an extended period of time for respiratory sound classification. The data may be processed into a spectrogram, and then a second algorithm may be used to analyze the spectrogram, in conjunction with other variables mentioned above. As a further example, the raw data may be used to improve the algorithm. For example, should an abnormal lung sound be recognized, it can serve as a control, and the raw data may be used as a dataset to further refine (or “train”) the pre-specified algorithm.

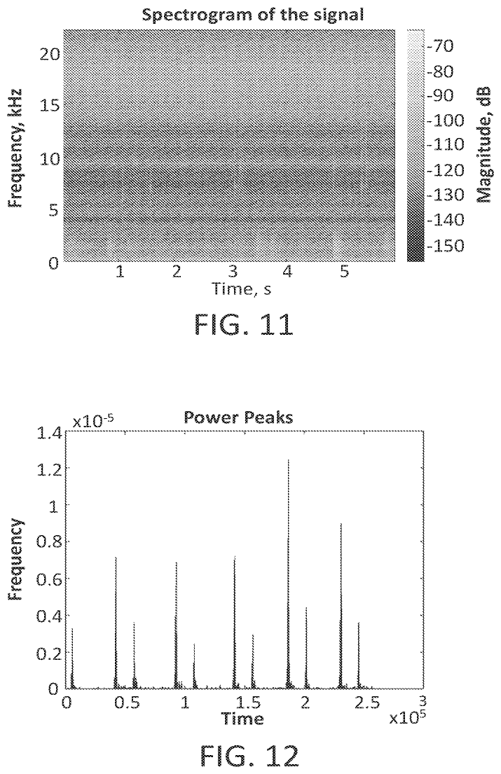

An exemplary spectrogram based on audio data captured in accordance with an exemplary embodiment is illustrated in FIG. 15. The top portion is obtained from a microphone facing towards the patient. The bottom portion is obtained from a microphone facing away from the patient.

Additional algorithms can be implemented in accordance with goals of the analysis. For example, in one embodiment, multiple sound samples are obtained and classified into different lung sounds. Next, the samples (spectrograms) are input into a pre-specified classification algorithm to generate a set of mathematical features. The difference between the output of this classification algorithm and the pre-defined mathematical features is used to refine the algorithms. The goal is ensure the classification algorithm has the variables needed to filter out unwanted noises during feature extraction.

Next, the classification algorithm can be applied to additional samples containing both an audio spectrogram and additional user data defined as “boundary conditions” above. The machine learning approach in this case need not focus on feature extraction. Rather, this machine learning approach employs predictive statistical analysis. The basic concept remains the same: the difference between the classification algorithm and the pre-defined answer is used to create and adjust the weight of variables.

An algorithm in accordance with an exemplary embodiment may be based on specific approaches used to train the algorithm, and the algorithm itself.

To further clarify, in one exemplary embodiment, a respiratory condition is detected by identifying how many times a certain type of respiratory sound occurs during a time period (“frequency”). If the number of times the sound is identified in a time period goes past a threshold, then a signal is generated to indicate that an adverse respiratory condition has been detected (or that an adverse respiratory condition has gotten better or worse). By saying “goes past a threshold” what is included is meeting the threshold, going above the threshold, or going below the threshold, depending upon what adverse respiratory conditions are desired to be detected. In a further exemplary embodiment, the number of times a certain type of respiratory sound occurs in a first time period is compared with the number of times the certain type of respiratory sound occurs in a second time period (the first and second time periods may or may not be overlapping, the first and second time periods may or may not be equal). For example, the number of respiratory sounds in a first time period may be compared with the number of respiratory sounds in a second time period greater than the first time period. Comparisons may be with regard to frequency, power, location in the time frame being evaluated, and/or other criteria. In one exemplary embodiment, the first time period may be three hours and the second time period may be 18 hours. These time periods are merely exemplary.

In another exemplary embodiment, respiratory issues are identified based on frequency of audio signal (wheeze frequency ˜300-400 Hz) and the number of times an event occurs (frequency of the event itself).

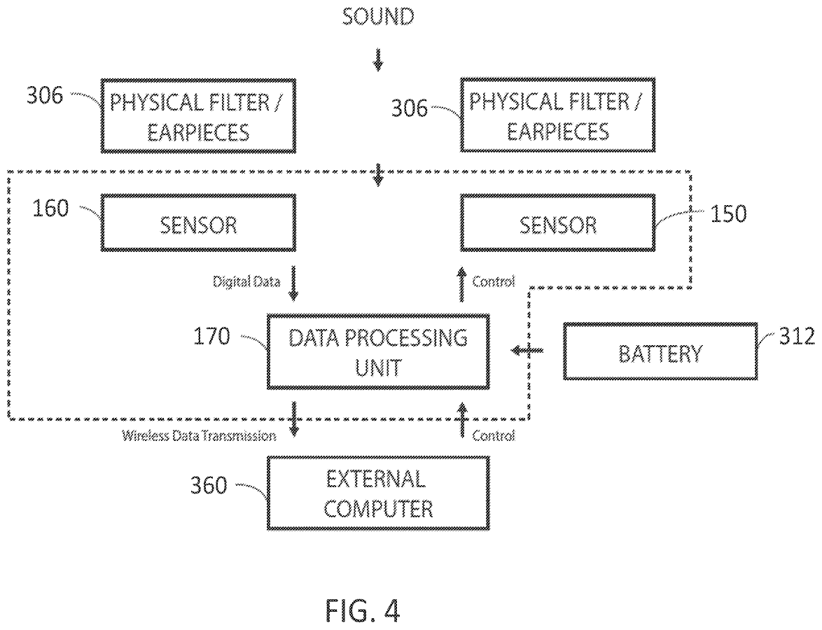

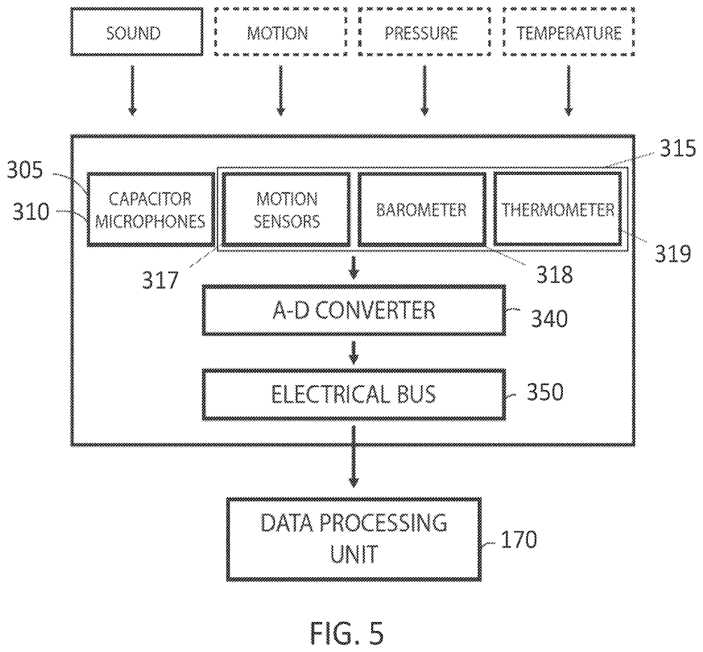

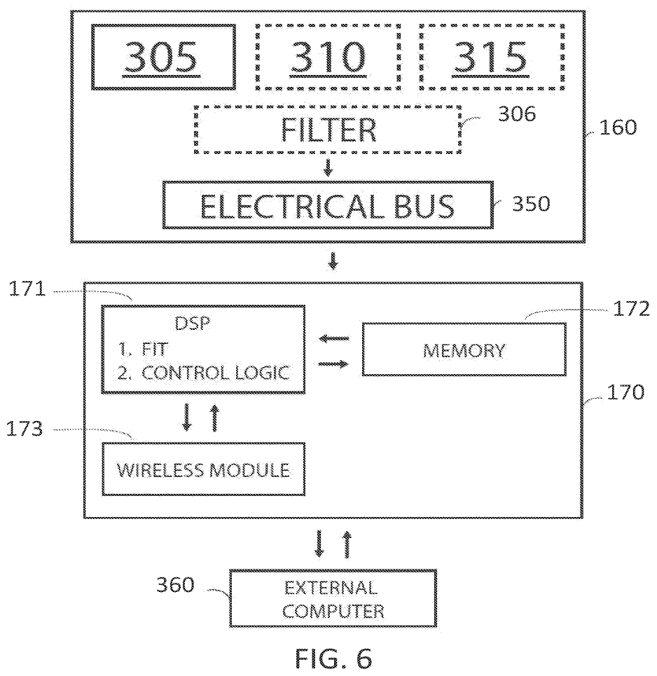

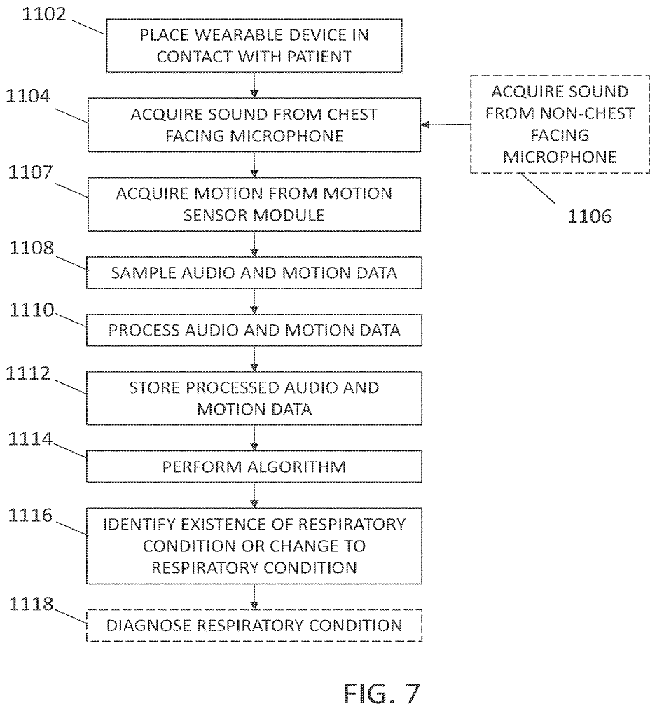

Alternatively, or additionally, the wearable device 100 can detect and monitor other physiological events. For example, the wearable device 100 can be used to detect heart rate and heart rate variability of the wearer. As described above, the wearable device 100 includes two microphones recording two channels of data. The first microphone 305 is facing the chest wall of the wearer and the second microphone 310 is facing away from the chest wall and is configured to capture primarily external sounds. FIG. 9 shows an exemplary sample of the two channels overlaid. In order to remove the external noise, the second signal is subtracted from the first signal. Next, a high pass filter is applied to the data, the result is shown in FIG. 10. FIG. 11 shows the same data in the form of a histogram. In the histogram, the high-volume peaks can be clearly seen. Finally, the data is squared to further highlight the heart beats detected by the first microphone 305, as shown in FIG. 12.

After filtering of the data, the peaks can be counted to determine a heart rate. A peak detection algorithm can be used to count the number of peaks at a predefined interval and store this value in a vector. The predefined interval can be any appropriate interval, such as 0.5 seconds. The vector of beats per interval can then be used to identify variability of the heart rate using root mean square of the successive differences method. The vector can also be used to calculate the average beats per minute.

In further embodiments, wearable device 100 may be configured to detect other heart sounds, such as heart murmurs and changes in the characteristics or rate of heart murmurs over time. The detection of heart sounds (e.g., using audio data from first microphone 305) along with activity and posture information derived from motion data captured by motion sensor module 317 may aid in the evaluation of diseases, including but not limited to diseases of the heart valve, heart failure, arrhythmias, and cardiac syncope. This may be especially helpful to monitor a patient at home, and to evaluate a patient's response to therapy at home.

In some embodiments, the presence of mouth-breathing can also be detected by comparing the audio data from first microphone 305 and second microphone 310. When the differential between lung sounds captured by first microphone 305 and second microphone 310 diminishes significantly, mouth breathing may be suspected. This is because the abnormal lung sounds can be transmitted to the ambient environment when the patient's mouth is open, and the sounds can subsequently be captured by the external microphone (e.g., second microphone 310). Mouth breathing is clinically significant as it may suggest deteriorating respiratory status in a patient. Further, the occurrence of mouth breathing in a patient that is also experiencing adventitious breath sounds in a stationary user (as determined based on data from motion sensor module 317) may indicate a user that is at risk. In such instances, an alert or other notification may be provided to the user or caregiver.

Further, a patient engaging in low-intensity ambulation (as determined by data from motion sensor module 317) who develops mouth breathing (whereas it was not present in prior days) indicate possible deteriorating disease and can serve as a trigger for further processing of the audio data, or provide another piece of input for processing (in combination with other inputs including lung sounds, chest wall movement, and inhaler use).

In another embodiment, the motion sensor module 317 is used to monitor additional physiological parameters. For example, the motion sensor module 317 can be used to monitor, for example, chest wall expansion, average tidal volume, respiratory rate, airflow rate, minute ventilation, and heart rate. These additional parameters can be important in evaluating patient health. For example, in some diseases tidal volume is a more reliable marker of pulmonary decompensation than respiratory rate.

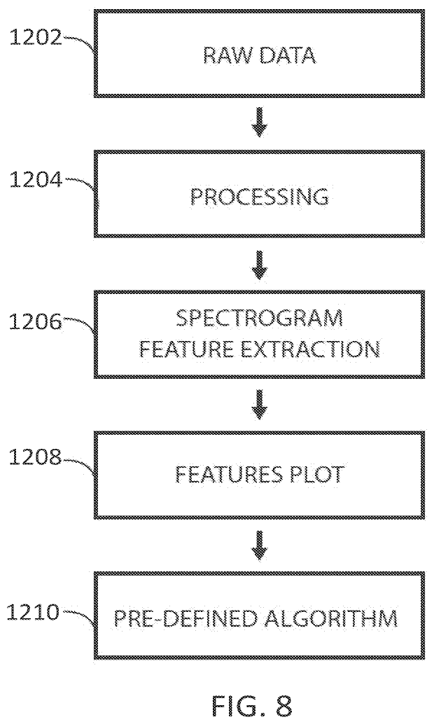

In one embodiment, the wearable device 100 is positioned at the point of maximum impulse (PMI) (i.e., the position at which oscillatory motion of the chest due to heart beat is most prominent). Alternatively, the motion sensor module 317 can be used to detect heart rate via ballistocardiography when the device is not placed near the PMI. As mentioned above, the motion sensor module 317 can include one or more accelerometers, a magnetometer, and a gyroscope. The signal from each of these sensors can be converted to standard units (e.g., m/s²) and summed. A low pass filter is applied to the data. FIG. 13 shows exemplary raw summed data and the data after the low pass filter is applied.

Respiration information can be determined by analyzing the data captured by the motion sensor module 317. A double integration method may be used to translate the accelerometer data into position data. After the raw acceleration and time data from the device is filtered and processed to display the correct units, it is integrated using the trapezoidal method of integration once to determine the velocity, then a second time to get a position vector. This position vector is then evaluated to find the individual breath waveforms.

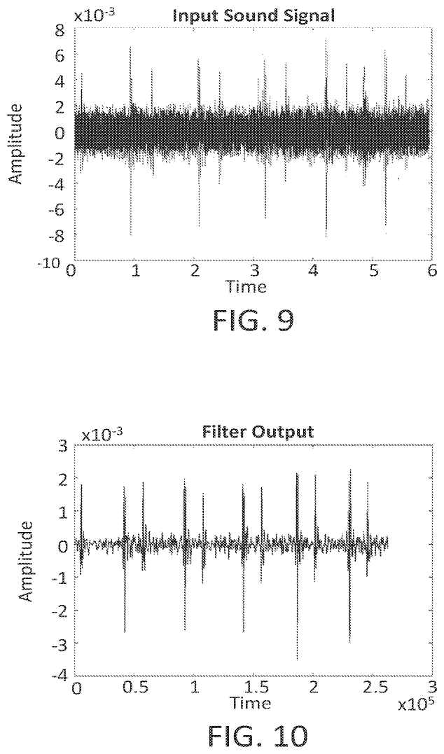

This position data can be used to determine tidal volume and chest wall expansion. For example, the data can be graphed. The peaks and valleys of the graphs correspond to the maximum volume and minimum volume, respectively, of the lungs. A peak locator function can used to locate the peaks. After identification of the peaks and valleys, the algorithm can split the data into separate breaths. The total distance traveled during each breath can then be calculated. An exemplary plot of a single breath is shown in FIG. 14.

The calculation of tidal volume can be further improved by using motion data captured by motion sensor module 317 in conjunction with audio data received from microphones 305, 310. For example, the amplitude of chest wall movement can be used to calculate the tidal volume, as described herein. In some embodiments, the reliability of this determination may be assessed based on respiratory sounds captured by, for example microphones 305, 310. The correlation of chest wall motion with tidal volume may be based on the assumption that the patient's airways are patent. As a result, if the patient's airways are not patent, the calculation of tidal volume based on chest wall motion may be inaccurate. Patency of the airway can be assessed by respiratory sounds. For example, chest wall movement that correlates with a tidal volume of 550 cc may be classified as accurate when respiratory sounds are normal (as determined by audio data captured by microphones 305, 310). The same chest wall movement, when associated with wheezes (as determined by audio data captured by microphones 305, 310) may be classified as less accurate. Similarly, the same chest wall movement may be classified as inaccurate when associated with absent of breath sounds (as determined by audio data captured by microphones 305, 310).

Additionally, in one embodiment the loudness of respiratory sounds may be correlated with the amount of air flow in the respiratory system. From the amount of flow and the duration of respiratory sounds, the tidal volume may be estimated. In such embodiments, the determination based on audio data may be compared with the determination based on chest wall movement to verify and/or adjust the calculation of tidal volume.

In addition, in some embodiments, the user wears more than one wearable device 100, allowing for more accurate calculation of the tidal volume. For example, in some embodiments, the user wears at least one device on each side of the user's torso. In some embodiments, one wearable device 100 is positioned on the anterior/superior chest wall and a second wearable device 100 is positioned on the xiphoid process of the user. The wearable device 100 on the anterior/superior chest wall may be best positioned to capture chest wall movement. The wearable device 100 positioned on the xiphoid process may be best positioned to capture different types of breathing styles, such as shallow breathing and belly breathing.

In some embodiments, the tidal volume (i.e., the amount of air that the patient moves in one minute) is also calculated based on the tidal volume and the rate of respiration. This may be done using both audio and motion data. A rapid increase or decrease in minute ventilation may indicate that the patient's condition is deteriorating and caregiver attention is required. In such instances, the wearable device 100 may issue or transmit an alert.

A heart beat can be distinguished from respiration based on the frequency of the signal and the magnitude of the movement of the chest wall. These differences are used to filter the signal to distinguish heart rate and respiration. The heartbeat waveforms can be isolated by correlating the vector magnitude among the three different sensors in the motion sensor module 317. The comparison of the waveforms of the individual sensors can be compared to identify the heart beats.

In addition to measuring and/or calculating linear displacement of the chest wall, angular displacement can be measured and/or calculated as well. The angular displacement can be used in addition to or as alternative to the linear displacement. The angular displacement can be determined based on a gyroscope of the motion sensor module 317.

The linear and/or angular velocity of the chest wall can also be used to determine the airflow rate.

Because the wearable device 100 detects both physiological sounds as well as movement of the chest wall, the accuracy of the identification of abnormalities and/or patterns in breathing can be improved. For example, the combination of motion sensors and microphones can be used to identify individuals with diminished breath sounds, such as those suffering from severe bronchospasm. The motion sensor module 317 can be used to identify phases in the respiratory cycle, as described above. Comparing the data gathered by the microphones during the various phases allows for more accurate identification of abnormalities in breath sounds.

Additionally, using the data from the motion sensor module 317 in conjunction with the data from the microphone(s) 305, 310 may allow for the differentiation of wheeze and stridor. These two conditions result in similar respiratory sounds. However, these sounds occur at different phases of the respiratory cycle. Hence, it may be difficult to differentiate these conditions using sound alone. However, by comparing the timing of the respiratory sounds with the chest wall movement data gathered by the motion sensor module 317, these conditions can be identified.

In one embodiment, the data gathered by the wearable device 100 is used to provide information regarding the patient during physical therapy. In such an embodiment, lung sound, chest wall motion, and other motion data including heart rate, posture, activity level, and gait are provided to the physical therapist or other caregiver via a software platform. Based on the data collected, real-time feedback and decision support is provided to the physical therapist for personalized therapy. Trending data can also be used to trend progress over time. This information can be used by the physical therapist to assess the patient's health and the efficacy of the physical training program. If necessary, the physical therapist can then make modifications to the training program. For example, if the patient's breathing is labored and/or abnormal, the physical therapist can reduce the intensity of the program. Alternatively, if the patient's breathing is within the desired range and is not indicative of an abnormality, the intensity of the program can be increased. The wearable device 100 may also allow the patient to safely perform training routines when the physical therapist is not present by providing continuous monitoring of the patient's breathing, heart rate, and other metrics. A physical therapist or physician can review this information, either during the exercise or at a later time, to ensure that the patient is not in danger.

The wearable device 100 can also be used to monitor compliance with prescribed or recommended activities. For example, incentive spirometry is often prescribed to prevent atelectasis in post-surgical patients. In one embodiment, the wearable device 100 includes a user interface that provides real-time feedback and instructions on prescribed rehab activities based on sensor data. Concurrently, sensor data can be sent to family members and clinical providers to monitor compliance and progress.

The microphones 305, 310 can also be used to detect other physiological events. In one embodiment, the wearable device 100 is placed on or near a major blood vessel. The wearable device 100 can detect the sound associated with blood flow through the blood vessel. The sound of blood flow through a blood vessel can be used to monitor narrowing of blood vessels, or “stenosis” of blood vessels, changes in the state of surgical stents, and changes in blood flow. The wearable device 100 can also detect the changes in the vibration of the skin surrounding the blood vessel, which correlates with the physiological state of the blood vessel wall, heart rate, and blood pressure, as well as the tissues that surround the blood vessel. Body sounds and motions then undergo processing by comparing the sounds to boundary conditions derived from predefined mathematical features derived from benchmark audio and motion data, as described above. This information can be used to diagnose or monitor vascular diseases, which include but are not limited to peripheral artery disease, carotid artery stenosis, abdominal aortic aneurysm, and access sites of endovascular procedures.

In another embodiment, the wearable device 100 is placed on or near a joint of the patient (e.g., the shoulder, the elbow, the hip, the knee, the ankle). The acoustic sound generated by the joint during movement is used to monitor orthopedic diseases. In one embodiment, a wearable device 100 is placed over more than one joint. For example, one wearable device can be placed over the left hip and one wearable device can be placed over the right hip. In such an embodiment, comparison of the data collected from the two devices allows for the identification of abnormalities in, for example, gait patterns. The identification can be performed by comparing the data collected to mathematical features derived from benchmark audio and motion data, as described above.

In another embodiment, the device is placed on the abdomen to detect abdominal sounds and abdominal movement. Acoustic analysis of abdominal sounds and the changes in abdominal movement undergo processing, as described above, to detect conditions that lead to fluids in the abdomen, rigidity of the abdominal wall, obstructions of the bowels, pseudo-obstructions of the bowels, and constipation.

In a further exemplary embodiment, the external computer (e.g., a smartphone, tablet computer, laptop computer, cloud-based computing system) modulates the frequency with which sensor 160 captures data.

The results of step 1118 (see FIG. 7) can be displayed and/or arranged in numerous manners. For example, it is possible to perform classification of audio data with boundaries set by user input. The classification can also be performed based on sensor data (i.e. gyroscope) included in a smartphone.

In one exemplary embodiment, a patient is able to provide feedback—i.e. a self-assessment of the diagnosis, in order to improve the accuracy of diagnosis. Regardless, historical data can be accumulated over periods of time (days, months, years) to further refine boundary conditions and models used to identify respiratory problems.

In one exemplary embodiment, a computing device other than a smartphone may be used. Exemplary computing devices include computers, tablets, etc.

In one exemplary embodiment, results of identification of respiratory illness, and/or changes in respiratory conditions, are provided to a patient provider. The identification and/or changes may be displayed using a variety of different user interfaces.

In one exemplary embodiment of the present invention, wearable device 100 provides an indication of remaining battery life.

In one exemplary embodiment, near-field communication (NFC) enabled tags are used to track medication and inhaler use. An NFC-enabled tag is attached to an inhaler or a medication container. After each use of the inhaler or each dose of medication, a user taps an NFC-enabled computing device to the NFC-enabled tag. The NFC-enabled computing device then records the time at which the tap occurs, which corresponds to the timing of the use of an inhaler or administering of a medication. The NFC-enabled computing device may include but is not limited to the following: mobile phone, tablet, or as part of the electronic components 130. The output of medication-use tracking is a “boundary condition” described above.

In one exemplary embodiment, results of identification and/or changes are pushed to a patient or to a patient provider. In another exemplary embodiment, results of identification and/or changes are pulled to a patient or to a patient provider (i.e. provided on demand).

In one exemplary embodiment, results of identification and/or changes are provided to a patient and/or patient provider in the form of emails and/or text messages and/or other forms of electronic communication. In one exemplary embodiment, the results are displayed in a software application (“app”) operating on a smartphone or other computing device.

The sampling frequency and sampling duration set forth above are merely exemplary. In one exemplary form of the present invention, sampling frequency and/or duration may be changed.

In one exemplary embodiment, the invention is used in combination with location technology such as GPS in order to locate location of a patient.

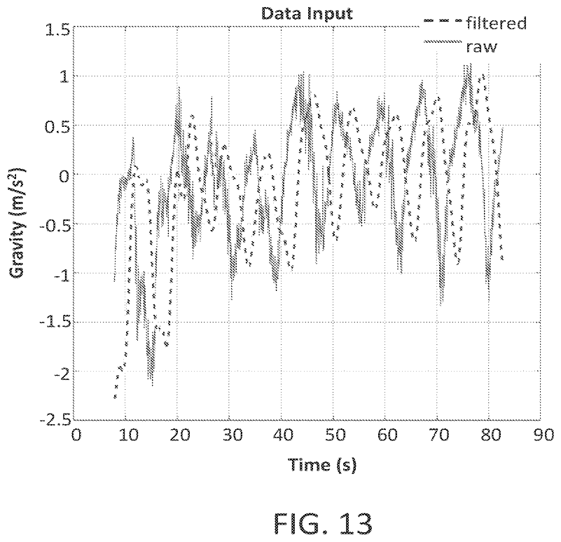

In one embodiment, shown in FIG. 16, a method of identifying physiological events is provided. The method includes affixing a wearable device to a user (step 1302). The wearable device includes at least one microphone, a motion sensor module, and a processor. The method further includes acquiring recorded audio data from the at least one microphone and recorded motion data from the motion sensor module (step 1304). The method further includes filtering a set of predefined audio samples based on the recorded motion data to arrive at a set of benchmark audio samples (step 1306). The method further includes extracting a first set of mathematical features from the set of benchmark audio samples (step 1308). The method further includes extracting a second set of mathematical features from the recorded audio data (step 1310). The method further includes comparing the second set of mathematical features to the first set of mathematical features to determine whether a physiological event has occurred (step 1312).

In one embodiment, the set of predefined audio samples are recorded from multiple subjects.

In one embodiment, the method further comprises, when the comparing step determines that a physiological event has occurred, performing a verification of the determination based on a comparison of additional mathematical features extracted from the recorded audio data with additional mathematical features extracted from the benchmark audio samples.

In one embodiment, the at least one microphone includes a first microphone and a second microphone, the first microphone oriented toward the user and the second microphone oriented away from the user. In such an embodiment, the method further includes subtracting the signal from the second microphone from the signal generated by the first microphone prior to extracting the second set of mathematical features.

In one embodiment, the filtering step further includes filtering the predefined spectrograms based on user data. In such an embodiment, the user data is selected from the group consisting of surgical history, disease condition, medication use, demographics, user weight, and user height.

In one embodiment, the wearable device is affixed at the point of maximum impulse.

In one embodiment, the wearable device is affixed adjacent a joint of the user.

In one embodiment, the wearable device is affixed to the abdomen of the patient.

In one embodiment, the method further includes exporting the recorded audio data and the recorded motion data to a computing device and analyzing the recorded audio data and the recorded motion data using the computing device to verify the determination of whether the physiological event has occurred. In one such embodiment, the analyzing step includes analyzing the recorded audio data and the recorded motion data based at least partially on parameters not used in the comparing step.

In another aspect, a system for providing feedback on physiological events is provided. The system includes a wearable device and a computing device. The wearable device is configured to be worn by a patient and includes at least one microphone configured to capture recorded audio data. The wearable device also includes a motion sensor module configured to capture recorded motion data. The wearable device also includes a processor configured to determine whether a physiological event has occurred based on the recorded audio data and the recorded motion data and generate a signal when the physiological event has occurred. The computing device includes a display and is in communication with the wearable device. The computing device is configured to: (i) receive the recorded audio data from the wearable device; (ii) receive the recorded motion data from the wearable device; (iii) receive the signal from the processor; and (iv) provide a graphical user interface on the display indicating that the physiological event has occurred.

In one embodiment, the computing device is a smartphone.

In another embodiment, the computing device further includes a processor, the processor configured to analyze the recorded audio data and the recorded motion data based at least partially on parameters not used by the processor of the wearable device.

In another aspect, a non-transitory computer readable medium containing computer-executable programming instructions for performing a method of identifying physiological events is provided. The method includes acquiring recorded audio data from at least one microphone and recorded motion data from a motion sensor module, the at least one microphone and the motion sensor module being housed in a wearable device affixed to a user. The method also includes filtering a set of predefined audio samples based on the recorded motion data to arrive at a set of benchmark audio samples. The method also includes extracting a first set of mathematical features from the set of benchmark audio samples. The method also includes extracting a second set of mathematical features from the recorded audio data. The method also includes comparing the second set of mathematical features to the first set of mathematical features to determine whether a physiological event has occurred. The method also includes causing a graphical user interface to responsively display an indication that the physiological event has occurred.

In another aspect, a method for analyzing respiratory motion is provided. The method includes affixing a wearable device to a user. The wearable device includes a motion sensor module. The method further includes acquiring recorded motion data from the motion sensor module. The method further includes calculating the movement of the chest wall to determine tidal volume of a respiration cycle.

In another embodiment, the wearable device includes at least one microphone and the method further includes acquiring recorded audio data with the at least one microphone, the recorded audio data including respiratory sounds. The method also includes determining the phase of the respiratory cycle during which the respiratory sounds occur based on the recorded motion data.

In another aspect, a method of identifying physiological events is provided. The method includes affixing a wearable device to a user. The wearable device includes at least one microphone and a processor. The method further includes acquiring recorded audio data from the at least one microphone. The method further includes filtering a set of predefined audio samples based on user data to arrive at a set of benchmark audio samples. The method further includes extracting a first set of mathematical features from the set of benchmark audio samples. The method further includes extracting a second set of mathematical features from the recorded audio data. The method further includes comparing the second set of mathematical features to the first set of mathematical features to determine whether a physiological event has occurred.

In one embodiment, the user data is selected from the group consisting of surgical history, disease condition, medication use, demographics, user weight, and user height.

In another aspect, a method of identifying physiological events is provided. The method includes affixing a wearable device to a user. The wearable device includes at least one microphone and a processor. The method further includes acquiring recorded audio data from the at least one microphone. The method further includes extracting a first set of mathematical features from a set of benchmark audio samples. The method further includes applying an adjustment to the first set of mathematical features to determine adjusted mathematical features. The method further includes extracting a second set of mathematical features from the recorded audio data. The method further includes comparing the second set of mathematical features to the adjusted mathematical features to determine whether a physiological event has occurred.

In one embodiment, the wearable device includes a motion sensor module and the method includes acquiring recorded motion data from the motion sensor module. The method further includes using the recorded motion data to calculate the adjusted mathematical features.

In one embodiment, the adjusted mathematical features are calculated using user data. The user data selected from the group consisting of surgical history, disease condition, medication use, demographics, user weight, and user height.

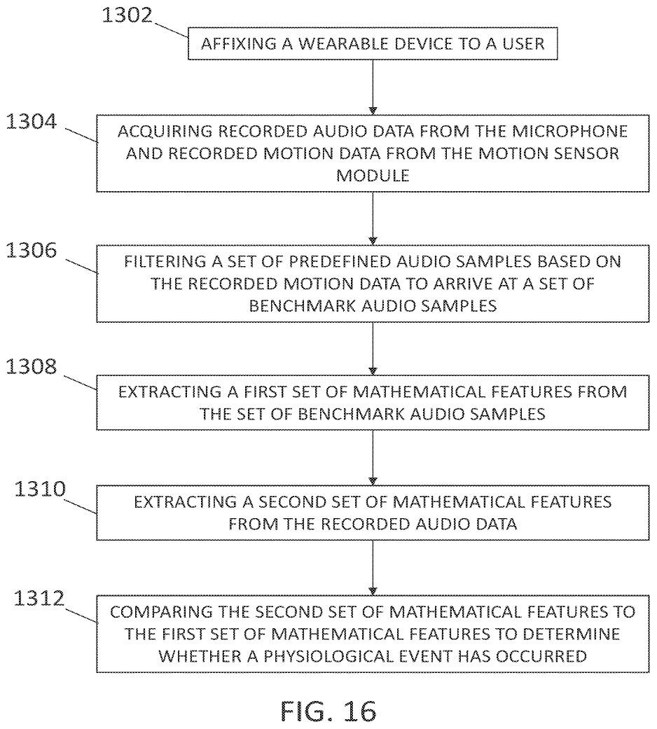

FIGS. 18 and 19 show methods of determining the aspiration risk associated with a cough detected using data gathered by wearable device 100. In FIG. 18, at step 1402, the cough is first detected based on audio using microphone 305 and/or microphone 310. The cough may be identified using any of the processes described herein. After identifying the cough, at step 1404, the user's chest wall movement is assessed. This assessment may be based on data received from motion sensor module 317. If the user's chest wall movement does not reflect that the user coughed, it may be determined that the user did not actually cough. For example, someone else in the area may have coughed or other ambient noises may have created the cough-indicative audio data. If, on the other hand, the motion data indicates that the chest wall did experience movement indicative of a cough, assessing the amplitude and/or acceleration of the chest wall movement. This may allow for a determination of whether the cough was a strong cough or a weak cough. For example, a high amplitude and/or acceleration of movement of the chest wall may indicate that it was a strong cough, with a corresponding low aspiration risk. On the other hand, a low amplitude and/or acceleration of chest wall movement may indicate a weak cough, with a corresponding higher aspiration risk. At step 1408, the respiratory pattern of the user may be assessed, based on motion data, to determine when in the respiratory cycle the cough occurred. This may further allow for a determination of the aspiration risk.

Turning to FIG. 19, at step 1502, the cough may be detected based on chest wall movement using motion data received from motion sensor module 317. For example, the cough may be identified by analysis of chest wall motion, velocity, acceleration, and derivatives thereof. After detecting a cough, at step 1504, the chest wall movement data may be assessed to determine if the cough was a strong cough or a weak cough. At step 1506, the audio data received from microphone 305 and/or microphone 310 may be analyzed. For example, if the analysis of the chest wall movement indicates that a strong cough has occurred, and the analysis of the audio data confirms this, it may be determined that a strong cough, with a low aspiration risk, has occurred. On the other hand, if the analysis of the chest wall movement indicates a strong cough, but the analysis of the audio data does not confirm a strong cough, this may indicate an obstruction of the user's upper airway. In such a scenario, the wearable device 100 may issue a notification to the user or a caregiver to check for an upper airway obstruction.

If the analysis of the chest wall movement indicates a weak cough and the audio data indicates a strong cough, this may be indicative of an error. For example, the wearable device 100 may be incorrectly positioned on the user's chest wall. If, instead, the analysis of the chest wall movement indicates a weak cough and the analysis of the audio data confirms this assessment, it may be determined that a weak cough has occurred. As described above, optionally, the respiratory pattern of the user may be assessed, based on motion data, to determine when in the respiratory cycle the cough occurred. This may further allow for a determination of the aspiration risk.

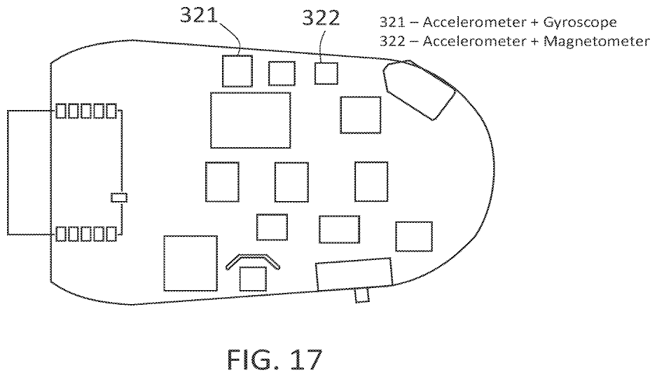

A method of determining the risk associated with a cough is shown in FIG. 20. At step 1602, a cough is detected. The cough may be detected through any of the processes described herein. For example, the cough can be detected by analyzing audio data received from microphone 305, 310 or motion data received from motion sensor module 317. At step 1604, the number of coughs occurring within a given interval is determined to identify clusters of coughs. For example, a cluster may be identified when three or more coughs are identified within 30 seconds. In other embodiments, different numbers of coughs or different durations (e.g., 10 seconds, 5 minutes, etc.) may be used to classify cough clusters. In some embodiments, the motion data can be used to identify cough clusters where an audio-based approach only identifies a single cough (i.e., when the patient's glottis is closed during a cough, or a loud ambient sound masks additional coughs). At step 1606, based on the frequency of the coughs, a risk level associated with the coughs is determined. At step 1608, based on this risk level, the threshold for activating further assessment algorithms may be adjusted. Assessing the risk in this way has a number of advantages. For example, by only implementing further assessment when a high-risk cluster of coughs is identified, battery and computing power may be conserved.

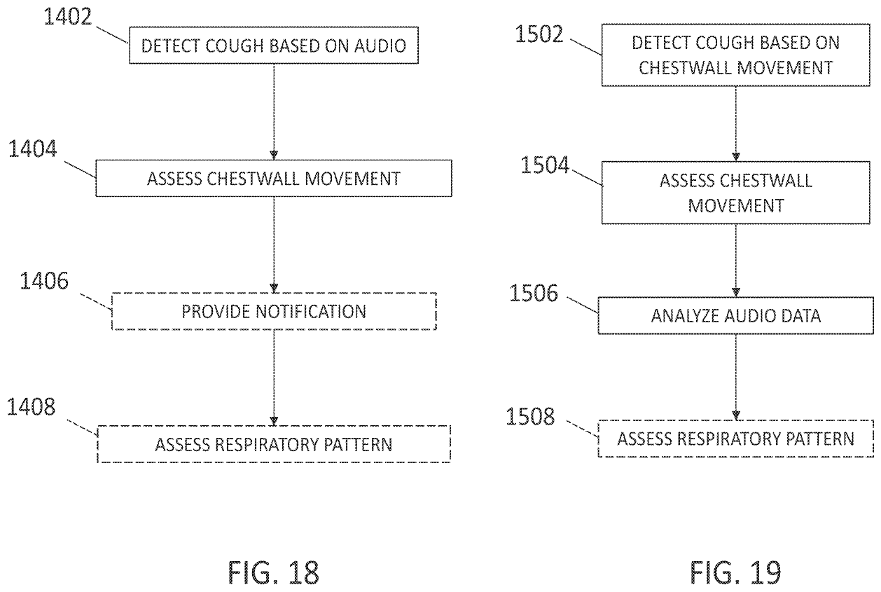

FIG. 21 illustrates a method of determining cough characteristics. At step 1702, a cough is detected. The cough may be detected through any of the processes described herein. For example, the cough can be detected by analyzing audio data received from microphone 305, 310 or motion data received from motion sensor module 317. At step 1704, the nature of the cough is determined (e.g., whether the cough is a dry cough or a wet cough). This may be done based on audio data received from microphone 305, 310, for example. In some embodiments, motion data received from motion sensor module 317 is used to determine whether the cough was a “strong” cough or a “weak” cough. Based on the nature and characteristics of the cough, an aspiration risk level may be determined. For example, a dry cough has a relatively lower risk of infection and/or aspiration, while a wet cough has a relatively higher risk of infection and/or aspiration. Based on the determination of the level of risk, at step 1710, further assessment algorithms may be initiated. By only initiating further assessment algorithms when a high-risk cough is detected, computing and battery resources may be conserved.

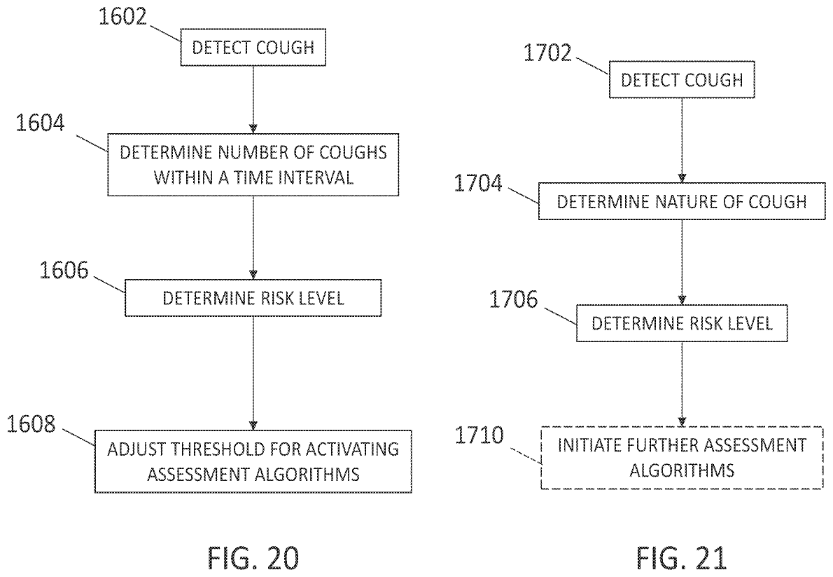

FIG. 22 illustrates another method of identifying a risk level associated with a cough. At step 1802, a cough is detected. The cough may be detected through any of the processes described herein. For example, the cough can be detected by analyzing audio data received from microphone 305, 310 or motion data received from motion sensor module 317. At step 1804, a determination is made of whether the cough rate has increased or decreased. For example, the number of coughs identified in the previous 24 hours may be compared with those received in the prior 72 hours. In addition, at step 1806, the user's activity level may be assessed based on motion data received from motion sensor module 317. If the rate of coughs has increased and the user's activity level has increased as well, the increased cough rate may be a result of exercised induced bronchospasm. In such a situation, no further action may be required. Further, if the cough rate has decreased and the activity level has increased, this may be an indication of improving symptoms. A decrease in cough rate and coincident decrease in activity level may indicate that there has not been a significant change in the user's symptoms.

In some embodiments, at step 1808, changes in the user's posture may be assessed using motion data received from motion sensor module 317. This may further assist with assessment of the user's condition. For example, if the user's cough rate has increased, the user's activity level has remained substantially the same or decreased, and the user's posture indicates that the user is lying down, this may indicate that the user is experiencing night time symptoms. In some instances, this may also indicate that the user is experiencing worsening heart failure. In instances in which the user's cough rate has increased, the user's activity level has remained the same or decreased, and the motion data indicates that the user is not lying down, this may be an indication that the user's symptoms are worsening. In some instances, this may also indicate that the user is experiencing worsening heart failure.

On the other hand, in instances in which the user's cough rate is decreasing, the user's activity level has remained substantially the same, and the user's posture has not changed, this may indicate that the user's symptoms are improving. In instances in which the user's cough rate is decreasing, the user's activity level has remained substantially the same, and the user's posture has not changed, this may indicate that the change in cough rate is posture related.

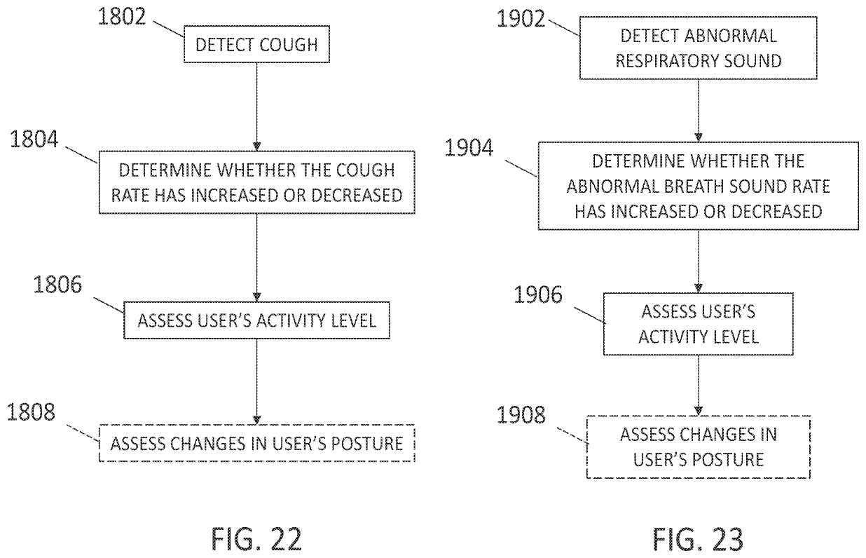

FIG. 23 illustrates a method for assessing the risk associated with an abnormal respiratory sound. The method includes many of the same processes and assessment as those described above with respect to FIG. 22. At step 1902, an abnormal respiratory sound may be detected. For example, the abnormal respiratory sound may be detected based on audio data received from microphone 305, 310. The abnormal respiratory sound may include, but is not limited to, a wheeze or rhonchi. At step 1904, it may be determined whether the rate at which the abnormal breath sound is occurring has increased or decreased. For example, the number of abnormal respiratory sounds identified in the previous 24 hours may be compared with those identified in the prior 72 hours. At step 1906, the user's activity level may be assessed based on motion data received from motion sensor module 317. Optionally, at step 1908, changes in the user's posture may be assessed based on motion data received from motion sensor module 317. Based on this information regarding the rate of abnormal respiratory sounds, the user's activity level, and changes in the user's posture, a risk level may be determined as described above with reference to FIG. 22. For example, in instances in which the rate of abnormal respiratory sounds has increased and the user's activity level has increased, this may indicate that the increased abnormal respiratory sound rate is related to exercise induced bronchospasm.

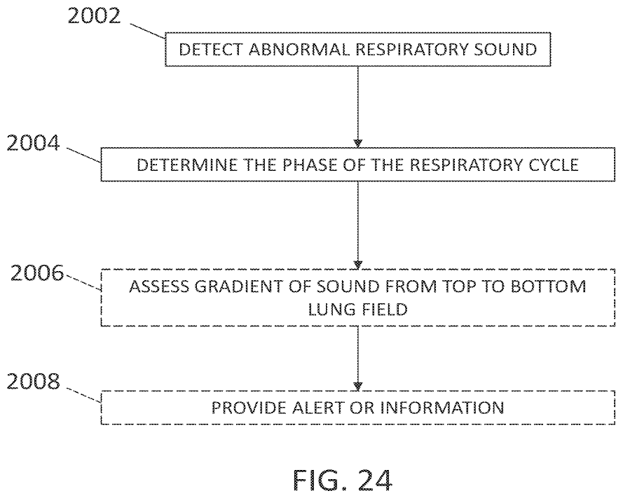

FIG. 24 illustrates another method of characterizing abnormal respiratory sounds, such as adventitious breath sounds. This may include, for example, wheezes, rhonchi, and rales. At step 2002, an abnormal respiratory sound may be detected using audio data received from microphone 305, 310. At step 2004, the phase of the respiratory cycle in which the abnormal respiratory sound occurred may be determined using motion data received from motion sensor module 317. In instances in which the abnormal respiratory sound occurs during the expiratory phase or both the expiratory and inspiratory phase of the respiratory cycle, the level of risk may be relatively low and information to be reviewed by a clinician may be generated, at step 2008.

On the other hand, if the user is wearing multiple devices (e.g., a first device and a second device) in instances in which the abnormal respiratory sound occurs during the inspiratory phase, it may be determined, at step 2006, whether there is a gradient between the upper and lower lung field. If there is no such gradient, or the gradient is low, the risk level may be relatively low, and information may be generated for a clinician to review, at step 2008. On the other hand, if there is a significant gradient between the upper and lower lung fields, this may indicate that the user has experienced a stridor. In such instances, an alert may be generated to make the user or a caregiver aware of the risk. The alert may be, for example, an audible alert or a tactile alert (e.g., vibration). Alternatively, or additionally, a text message, email, or other text-based alert may be generated and transmitted to the user, a caregiver, or a clinician.

In some instances, the abnormal respiratory sound identified using the audio data is an adventitious breath sound (e.g., wheezes, rhonchi, whistles, etc.). In other instances, the abnormal respiratory sound is indicative of the user's use of an inhaler. In such instances, the audio data can be used to determine the type of inhaler being used. This may be done using audio data received from the chest facing microphone 305 as well as the background microphone 310. Different types of inhalers lead to different types of sounds that can be identified in the audio data. Further, the audio data can be analyzed to identify lung sounds occurring during inhaler use. Further, the motion data can be analyzed to determine in which phase of the respiratory cycle the inhaler is used (e.g., based on chest wall movement).

The analysis of the user's use of the inhaler may be used to identify incorrect inhaler use. Many patients employ the wrong technique when using their inhalers, leading to suboptimal dosage. Deviation from normal inhaler sound and chest wall movement can be used to identify inhaler misuse. Specifically, the timing of inhaler “clicks” and/or the timing of respiratory sounds indicative of inhaler use as compared to chest wall movements, could be used to identify inhaler misuse.

The methods described above and illustrated in FIGS. 18-24 each provide examples of the advantages provided by monitoring and analyzing both audio data received from microphone 305, 310 and motion data received from motion sensor module 317. The ability to analyze coughs and other abnormal respiratory sounds using sounds generated in the respiratory system along with, for example, chest wall movement, user activity level, and user posture allows for the analysis of risk levels that are not possible using audio alone. The use of both motion data and audio data may improve the accuracy of classifying a specific type of respiratory sound and allow for further characterization of a specific type of respiratory sound. This provides significant clinical advantages. By including both an audio sensor, such as a microphone, along with one or more motion sensors in the same wearable device, reliable data can be captured and analyzed.

While the foregoing description and drawings represent preferred or exemplary embodiments of the present invention, it will be understood that various additions, modifications and substitutions may be made therein without departing from the spirit and scope and range of equivalents of the accompanying claims. In particular, it will be clear to those skilled in the art that the present invention may be embodied in other forms, structures, arrangements, proportions, sizes, and with other elements, materials, and components, without departing from the spirit or essential characteristics thereof. One skilled in the art will further appreciate that the invention may be used with many modifications of structure, arrangement, proportions, sizes, materials, and components and otherwise, used in the practice of the invention, which are particularly adapted to specific environments and operative requirements without departing from the principles of the present invention. The presently disclosed embodiments are therefore to be considered in all respects as illustrative and not restrictive, the scope of the invention being defined by the appended claims and equivalents thereof, and not limited to the foregoing description or embodiments. Rather, the appended claims should be construed broadly, to include other variants and embodiments of the invention, which may be made by those skilled in the art without departing from the scope and range of equivalents of the invention. All patents and published patent applications identified herein are incorporated herein by reference in their entireties.

1-21. (canceled)

22. A method for acquiring chest sound information from a first patient, comprising:
a) acquiring, by a wearable device, chest sound data from the first patient, wherein:
i) the device is configured with a microphone and a motion sensor; and
ii) during acquisition of the first patient chest sound data:
(1) the device is affixed to the patient's chest with an adhesive; and
(2) the microphone is arranged to face the first patient's chest;
b) acquiring motion data from the first patient via a motion sensor module configured with the device;
c) processing the first patient chest sound data to generate chest sound spectrogram data for the first patient;
d) comparing the first patient chest sound spectrogram data with a collection of predefined chest sound spectrogram data;
e) identifying one or more abnormal lung sounds in the first patient chest sound data, wherein the identified abnormal lung sounds occur during one or more first patient respiratory cycles;
f) processing the first patient motion data to determine where in the one or more first patient respiratory cycles the identified one or more patient abnormal lung sounds occurred;
g) generating first patient chest sound information associated with both the one or more identified abnormal lung sounds and where in the one or more patient respiratory cycles the abnormal lung sound occurred; and
h) providing the first patient chest sound information to the first patient, a physician, or a caregiver.

23. The method of claim 22, wherein the microphone is incorporated in a housing.

24. The method of claim 22, wherein the first patient chest sound data is filtered to distinguish lung sounds from non-lung sounds.

25. The method of claim 22, wherein the predefined spectrogram data is generated by:
a) acquiring chest sound data from a plurality of persons other than the first patient;
b) processing the chest sound data to generate a plurality of chest sound spectrograms associated with persons other than the first patient;
c) identifying abnormal lung sounds in the chest sound data associated with the persons other than the first patient; and
d) correlating the identified abnormal lung sounds with a corresponding chest sound spectrogram, thereby providing a collection of predefined chest sound spectrograms generated from persons other than the first patient.

26. The method of claim 22, wherein the predefined spectrogram data is generated by:
a) acquiring historical chest sound data from the first patient at a time period prior to the acquisition of the chest sound data;
b) processing the historical chest sound data to generate historical lung sound spectrogram data associated with the first patient;
c) identifying chest sounds in the historical first patient chest sound data; and
d) correlating the identified historical chest sounds with a corresponding historical chest sound spectrogram, thereby providing historical chest sound spectrogram data for the first patient.

27. The method of claim 25, wherein the one or more abnormal lung sounds associated with the predefined spectrogram data are identified by a physician.

28. The method of claim 22, further comprising:
a) generating a boundary condition for the first patient chest sound information; and
b) providing an alert when the first patient chest sound information exceeds the boundary condition.

29. The method of claim 22, wherein the one or more abnormal lung sounds comprise adventitious lung sounds.

30. The method of claim 22, wherein the identified abnormal lung sound comprises a crackle, a wheeze, a rhonchi, a stridor, or labored breathing.

31. The method of claim 22, wherein the predefined chest sound spectrograms are selected to evaluate a medical condition associated with the first patient.

32. The method of claim 22, wherein the first patient chest sound information includes detection of medical issues associated with respiratory activity of the first patient.

33. The method of claim 22, further comprising processing the first patient chest sound data to filter sounds other than lung sounds.

34. The method of claim 22, wherein the first patient chest sound data and motion data are processed along with additional first patient information.

35. The method of claim 34, wherein the additional first patient information comprises first patient medical records.

36. The method of claim 34, wherein the additional first patient information comprises one or more of:
a) demographics;
b) medication usage;
c) oxygen saturation;
d) fluid intake
e) fluid output
f) surgical history;
g) physical activity level;
h) weight;
i) fluid intake/output; or
j) physical activity level.

37. The method of claim 22, wherein the motion sensor module comprises at least one of:
a) an accelerometer;
b) a gyroscope; or
c) a magnetometer.

38. The method of claim 22, wherein the motion data provides information associated with one of more of:
a) a positioning of the first patient when the one or more abnormal lung sounds are detected;
b) whether the first patient was coughing or when the one or more abnormal lung sounds are detected; or
c) a correlation between the first patient's breathing irregularities and positional changes.

39. The method of claim 22, wherein the motion sensor module is configured to sense both linear and angular motion of the patient's chest wall during a first patient respiratory cycle.

40. The method of claim 22, wherein the motion sensor module is configured to monitor one or more of:
a) chest wall expansion;
b) average tidal volume;
c) respiratory rate;
d) airflow rate;
e) minute ventilation; or
f) heart rate.

41. The method of claim 22, wherein the motion sensor module is configured to determine characteristics of a respiratory cycle associated with the first patient comprising one or more of:
a) an amplitude of a breath;
b) a duration of inhalation and exhalation; or
c) a duration of an interval between a first breath and a second breath.
